(19) United States
(12) Patent Application Publication
(10) Pub. No.: US 2025/0313854 A1
(43) Pub. Date: Oct. 9, 2025

(54) METHOD FOR PRODUCING RECOMBINANT AAV PARTICLES

(71) Applicant: Hoffmann-La Roche Inc., Little Falls, NJ (US)

(72) Inventors: Sven Markert, Sindelsdorf (DE); Elisabeth Zollbrecht, Kochel am See (DE); Detlef Eisenkraetzer, Iffeldorf (DE)

(73) Assignee: Hoffmann-La Roche Inc., Little Falls, NJ (US)

(21) Appl. No.: 18/965,956

(22) Filed: Dec. 2, 2024

Related U.S. Application Data
(63) Continuation of application No. PCT/EP2023/064647, filed on Jun. 1, 2023.

(30) Foreign Application Priority Data
Jun. 3, 2022 (EP) 22177118.1

(51) Int. Cl. C12N 15/86

(52) U.S. Cl. CPC C12N 15/86; C12N 2750/14143; C12N 2750/14151

(57) ABSTRACT

Herein is reported a method for producing a recombinant AAV particle comprising the steps of propagating a mammalian cell using perfusion until at least a first pre-determined cell density is obtained; diluting an aliquot the propagated cells by adding fresh cultivation medium to obtain a production cell solution that has a second pre-determined cell density; cultivating the production cell solution for 1 to 36 hours; transfecting the cells directly in the cultivated production cell solution with one or more nucleic acids encoding for the recombinant AAV particle; and cultivating the transfected production cell solution for 24 to 144 hours thereby producing a recombinant AAV particle.

### CROSS-REFERENCE TO RELATED APPLICATIONS

This application is a continuation of International Application No. PCT/EP2023/064647, filed Jun. 1, 2023, which claims benefit of priority to European Patent Application No. 22177118.1 filed Jun. 3, 2022, each of which is incorporated herein by reference in its entirety.

### FIELD OF THE INVENTION

The current invention is in the field of gene therapy. More precisely herein is reported a method for the production of recombinant AAV particles, wherein the cells have been cultivated using perfusion prior to transfection and transient production of the recombinant AAV particle, which is performed without perfusion.

### BACKGROUND

Gene therapy refers broadly to the therapeutic administration of genetic material to modify gene expression of living cells and thereby alter their biological properties. After decades of research, gene therapies have progressed to the market and are expected to become increasingly important. In general, gene therapy can be divided into either in vivo or ex vivo approaches.

Today, most in vivo therapies rely on DNA delivery with recombinant adeno-associated viral (rAAV) vectors. An AAV is a small, naturally occurring, non-pathogenic parvovirus, which is composed of a non-enveloped icosahedral capsid. It contains a linear, single stranded DNA genome of approximately 4.7 kb. The genome of wild-type AAV vectors carries two genes, rep and cap, which are flanked by inverted terminal repeats (ITRs). ITRs are necessary in cis for viral replication and packaging. The rep gene encodes for four different proteins, whose expression is driven by two alternative promoters, P5 and P19. Additionally different forms are generated by alternative splicing. The Rep proteins have multiple functions, such as, e.g., DNA binding, endonuclease and helicase activity. They play a role in gene regulation, site-specific integration, excision, replication and packaging. The cap gene codes for three capsid proteins and one assembly-activating protein. Differential expression of these proteins is accomplished by alternative splicing and alternative start codon usage and is driven by a single promoter, P40, which is located in the coding region of the rep gene.

In engineered, therapeutic rAAV vectors, the viral genes are replaced with a transgene expression cassette, which remains flanked by the viral ITRs, but encodes a gene of interest under the control of a promoter of choice. Unlike the wild-type virus, the engineered rAAV vector does not undergo site-specific integration into the host genome, remaining instead predominantly episomal in the nucleus of transduced cells.

An AAV is not replication competent by itself but requires the function of helper genes. These are provided in nature by co-infected helper viruses, such as, e.g., adenovirus or herpes simplex virus. For instance, five adenoviral genes, i.e. E1A, E1B, E2A, E4 and VA, are known to be essential for AAV replication. In contrast to the other helper genes, which code for proteins, VA is a small RNA gene.

For the production of rAAV vectors, DNA carrying the transgene flanked by ITRs is introduced into a packaging host cell line, which also comprises rep and cap genes as well as the required helper genes. There are many ways of introducing these three groups of DNA elements into cells and ways of combining them on different DNA plasmids (see, e.g., Robert, M. A., et al. Biotechnol. J. 12 (2017) 1600193).

Two general production methods are widely used. In the triple transfection method, a plasmid comprising rep/cap and a plasmid comprising the rAAV-transgene are transiently co-transfected with an adenovirus helper plasmid carrying the required adenoviral helper genes. The process can be performed using CHO or HEK cells. Alternatively, rep/cap and viral helper genes can be combined on one larger plasmid (dual transfection method). The second method encompasses the infection of insect cells (Sf9) with two baculoviruses, one carrying the rAAV genome and the other carrying rep and cap. In this system, helper functions are provided by the baculovirus plasmid itself. In the same way, herpes simplex virus is used in combination with HEK293 cells or BHK cells. More recently Mietzsch et al. (Hum. Gene Ther. 25 (2014) 212-222; Hum. Gene Ther. Methods 28 (2017) 15-22) engineered Sf9 cells with rep and cap stably integrated into the genome. With these cells a single baculovirus carrying the rAAV transgene is sufficient to produce rAAV vectors. Clark et al. (Hum. Gene Ther. 6 (1995) 1329-1341) generated a HeLa cell line with rep/cap genes and a rAAV transgene integrated in its genome. By transfecting the cells with wild-type adenovirus, rAAV vector production is induced and mixed stocks of rAAV vectors and adenovirus are produced.

Perfusion culture has been reported to be applicable in classical recombinant protein production (see Woodgate, J. N., in “Biopharmaceutical Processing: Development, Design and Implementation of Manufacturing Processes” (2018), pages 755-768). For mammalian cell lines in such classical recombinant protein production, cell cultures are taking between 10 and 14 days on average for stable recombinant protein products, wherein the production bioreactor is the end of a long train of seed bioreactors of 1000 L, 100 L, 10 L and 1 L in scale to generate a sufficient starting number of cells with which are used to inoculate the production bioreactor. The cells are constantly being diluted with new cell culture media before mid-exponential growth has been reached. This ensures that the cells can be maintained in exponential growth as they are not exposed to nutrient-limiting (e.g., where growth associated amino acids are limiting) or toxic environments (e.g., where metabolic byproduct such as lactate and ammonia accumulate to high levels).

Yang, W. C., et al. (Biotechnol. Prog. 30 (2014) 616-625) showed proof-of-concept for an Alternating Tangential Flow (ATF) perfusion seed cultivation used for a subsequent standard fed-batch process by using a high cell density seed culture (400*10\hat{ }5 cell/mL) in order to inoculate the production bioreactor at a much higher concentration (standard 100*10\hat{ }5 cells/mL). They completed this study with two CHO cell lines expressing monoclonal antibodies and showed that both achieved their typical 5 g/L product titers yet in 12 rather than 17 days, thus increasing manufacturing capacity by 30% while maintaining (and for one cell line improving) the product quality profile.

In U.S. Pat. No. 6,566,118 methods and compositions for producing high titer, substantially purified preparations of recombinant adeno-associated virus (AAV) that can be used as vectors for gene delivery are reported.

In WO 2020/154607 are reported methods of producing adeno-associated virus (AAV) comprising culturing AAV producer cell lines in a seed culture followed by an AAV production culture.

### SUMMARY OF THE INVENTION

Herein is reported a method for the production of recombinant AAV particles using mammalian cells, wherein the mammalian cells
i) have been propagated prior to the AAV particle production in a cultivation with perfusion,
ii) have been diluted/split to a lower cell density after the propagation, and
iii) are cultivated after transfection with the nucleic acids required for the production of the recombinant AAV particle without perfusion.

The invention is based, at least in part, on the finding that it is advantageous to propagate mammalian cells, which are intended for the production of recombinant AAV particles, prior to the actual recombinant AAV particle production, even prior to the transfection with the nucleic acids required for the production of the recombinant AAV particle, at least some time in a perfusion cultivation and split the cells/dilute with fresh medium thereafter but prior to the transfection with the nucleic acids required for recombinant AAV particle production.

It has been found that by using mammalian cells propagated according to the invention for the production of recombinant AAV particles, the viable cell density in the production cultivation is higher compared to a production cultivation in which cells are used that have been propagated using fed-batch only when starting from the same inoculation cell density. The cells even further propagate for a short period after the transfection, i.e. the cell density increases after the transfection. Without being bound by this theory, it is assumed that this is a result of the better metabolic condition of cells derived from a process using N-1 perfusion. Consequently, the cells can better resist stress resulting from transfection, which results in a higher recombinant AAV particle yield.

In one aspect, a method for producing a recombinant AAV particle includes the following steps a) propagating a mammalian cell using perfusion until at least a first pre-determined cell density is obtained/achieved, b) diluting an aliquot/a fraction of the cells obtained in step a) by adding not-used/fresh cultivation medium to obtain a production cell solution that has a second pre-determined cell density, c) cultivating the production cell solution for 1 to 36 hours, d) transfecting the cells directly in the cultivated production cell solution obtained in step c) with one or more nucleic acids encoding for the recombinant AAV particle, e) cultivating the transfected production cell solution obtained in step d) for 24 to 96 hours, thereby producing a recombinant AAV particle.

The invention encompasses the following independent aspects and dependent embodiments:
1. A method for producing a recombinant AAV particle comprising the following steps:
   a) propagating a mammalian cell using perfusion until at least a first pre-determined cell density is obtained/achieved;
   b) diluting an aliquot/a fraction of the cells obtained in step a) by adding not-used/fresh cultivation medium to obtain a production cell solution that has a second pre-determined cell density;
   c) cultivating the production cell solution for 1 to 36 hours;
   d) transfecting the cells directly in the cultivated production cell solution obtained in step c) with one or more nucleic acids encoding for the recombinant AAV particle;
   e) cultivating the transfected production cell solution obtained in step d) for 20 to 240 hours; thereby producing a recombinant AAV particle.
2. The method of aspect 1, wherein the first pre-determined cell density is at least 80*10\hat{ }5 cells/mL.
3. The method of aspect 1 and embodiment 2, wherein the first pre-determined cell density is at least 100*10\hat{ }5 cells/mL.
4. The method of aspect 1 or any one of embodiments 2 to 3, wherein the mammalian cell is a CHO cell or a HEK cell or a human amniocyte cell.
5. The method of aspect 1 or any one of embodiments 2 to 4, wherein the mammalian cell is a CHO-K1 or a HEK293 cell.
6. The method of aspect 1 or any one of embodiments 2 to 5, wherein the one or more nucleic acids comprise
   i) a transgene comprising in 5′- to 3′-direction:
      alpha) a first ITR sequence;
      beta) a promoter;
      gamma) a nucleic acid sequence encoding a therapeutic molecule;
      delta) a polyadenylation signal sequence;
      epsilon) a second ITR sequence;
   ii) a rep open reading frame;
   iii) a cap open reading frame; and
   iv) adenoviral E1A, E1B, E2A, E4orf6, and VA RNA open reading frames.
7. The method of aspect 1 or any one of embodiments 2 to 6, wherein in step b) 4-times to 6-times the volume of the aliquot of fresh cultivation medium is added.
8. The method of aspect 1 or any one of embodiments 2 to 7, wherein in step b) about 5-times the volume of the aliquot of fresh cultivation medium is added.
9. The method of aspect 1 or any one of embodiments 2 to 8, wherein the second pre-determined cell density is between 10*10\hat{ }5 cells/mL and 30*10\hat{ }5 cells/mL.
10. The method of aspect 1 or any one of embodiments 2 to 9, wherein the second pre-determined cell density is about 20*10\hat{ }5 cells/mL.
11. The method of aspect 1 or any one of embodiments 2 to 10, wherein the cultivating in step c) is for 16 to 30 hours.
12. The method of aspect 1 or any one of embodiments 2 to 11, wherein the cultivating is for 20 to 28 hours.
13. The method of aspect 1 or any one of embodiments 2 to 12, wherein the cultivating is for about 24 hours.
14. The method of aspect 1 or any one of embodiments 2 to 13, wherein the cultivating in step e) is a batch cultivation.
15. The method of aspect 1 or any one of embodiments 2 to 14, wherein the cultivating in step e) is for about 24 to 144 hours.
16. The method of aspect 1 or any one of embodiments 2 to 15, wherein the cultivating in step e) is for about 48 to 120 hours.
17. The method of aspect 1 or any one of embodiments 2 to 16, wherein the cultivating is for about 60 to 96 hours.
18. The method of aspect 1 or any one of embodiments 2 to 17, wherein the cultivating is for about 72 hours.
19. The method of aspect 1 or any one of embodiments 2 to 18, wherein step e) is without feeding.
20. The method of aspect 1 or any one of embodiments 2 to 19, wherein the method further comprises the steps of
   f) harvesting the cells from the cultivated solution of step e);
   g) lysing the cells obtained in step f);
   h) separating the AAV particles from the lysed cells obtained in step g);
   i) optionally purifying the AAV particles.
21. The method of aspect 1 or any one of embodiments 2 to 20, wherein the cell density after step c) is 35-40*10\hat{ }5 cells/ml.
22. The method of aspect 1 or any one of embodiments 2 to 21, wherein after step c) and prior to step d) the following step cd) is performed:
   cd) adding an additional about 20% of the initial cultivation volume fresh medium.
23. The method of aspect 1 or any one of embodiments 2 to 22, wherein the transfecting is by adding the one or more nucleic acids as a mixture of PEI-complexed nucleic acid and free PEI.
24. The method of aspect 1 or any one of embodiments 2 to 23, wherein the transfecting is in the presence of a final concentration of 5 mM valproic acid.
25. Use of a mammalian cell for the production of recombinant AAV particles, wherein the mammalian cell has been propagated using perfusion prior to transfection with all AAV particle encoding nucleic acids.
26. Use of a mammalian cell obtained with a method according to aspect 1 or any one of embodiments 2 to 24 for the production of recombinant AAV particles.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Viable cell density in fed-batch, N-2 (blue); fed-batch, N-1 (green); production stage, N (red) cultivations.

FIG. 2 Viable cell density N-2, fed-batch, 10 L scale, N-2 (blue); non-controlled perfusion, 25 L scale, N-1 (green); production stage, 100 L scale, N (red) cultivations.

FIG. 3 Viable cell density of 1 L scale, N-1, controlled perfusion cultivation.

FIG. 4 Viable cell density for VCD-controlled perfusion in 100 l scale: green=N-1, controlled perfusion; red=N, production stage cultivation.

FIG. 5 Viable cell density of the permittivity controlled perfusion process; N-1, permittivity-controlled perfusion cultivation.

FIG. 6 Production of an AAV particle of subtype 2 variant 7m8 comprising a transgene encoding a therapeutic Fab. Viable cell density of biomass-sensor-controlled perfusion process, N-1 (green); production stage, N (red) cultivation.

FIG. 7 Viable cell densities of VCD-controlled perfusion and biosensor-controlled perfusion cultivations: red (VCD) versus violet (biosensor); blue (VCD) versus green (Biomass-sensor)).

FIG. 8 Viable densities obtained in different production scale cultivations inoculated with cells obtained with perfusion in the N-1 stage, i.e. according to the invention, and without perfusion in the N-1 stage; red curves correspond to production scale cultivation with N-1 cultivations without perfusion and the green curves (marked as “1”, “2”, “3”, “4”) correspond to production scale cultivations with N-1 cultivations with perfusion according to the invention.

FIG. 9 Viability of the cells in the production scale cultivation N depending on the cultivation in the N-1 stage of the cells used for inoculation of the N stage. The red curves correspond to a production culture inoculated with cells obtained from a batch N-1 cultivation. The green curves (denoted as “1”, “2”, “3”, and “4”) correspond to production scale cultivations inoculated with cells obtained from N-1 cultivations with perfusion according to the current invention.

FIG. 10 Capsid titer in the production culture in bioreactor systems >10 L in N after N-1 with and without perfusion. The red curves (“5”, “6”) correspond to a production culture following a batch N-1. The green curves (“1”, “2”, “3”, and “4”) correspond to production cultures following N-1 with perfusion. For the red curves, batch culture N-1 was executed in the same bioreactor (therefore N scale cultivation day zero corresponds to day 4 in the figure)

### DETAILED DESCRIPTION OF SPECIFIC EMBODIMENTS OF THE INVENTION

### Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N. Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) TRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Deoxyribonucleic acids comprise a coding and a non-coding strand. The terms “5′” and “3′” when used herein refer to the position on the coding strand.

The term “3′ flanking sequence” denotes a sequence located at the 3′-end (downstream of, below) a nucleotide sequence.

The term “5′ flanking sequence” denotes a sequence located at the 5′-end (upstream of, above) a nucleotide sequence.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “AAV helper functions” denotes AAV-derived coding sequences (proteins) which can be expressed to provide AAV gene products and AAV particles that, in turn, function in trans for productive AAV replication and packaging. Thus, AAV helper functions include AAV open reading frames (ORFs), including rep and cap and others such as AAP for certain AAV serotypes. The rep gene expression products have been shown to possess many functions, including, among others: recognition, binding and nicking of the AAV origin of DNA replication; DNA helicase activity; and modulation of transcription from AAV (or other heterologous) promoters. The cap gene expression products (capsids) supply necessary packaging functions. AAV helper functions are used to complement AAV functions in trans that are missing from AAV vector genomes.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “batch culture” refers to a culture in which all components for cell culturing (including the cells and all culture nutrients) are supplied to the culturing bioreactor at the start of the culturing process.

The terms “comprise(s),” “include(s),” “having,” “has,” “can,” “contain(s)” and variants thereof, as used herein, are intended to be open-ended transitional phrases, terms or words that do not preclude the possibility of additional acts or structures. The term “comprising” also encompasses the term “consisting of”. The present disclosure also contemplates other embodiments “comprising,” “consisting of” and “consisting essentially of,” the embodiments or elements presented herein, whether explicitly set forth or not.

The term “cultivate” herein refers to the step of maintaining cells in a cultivation medium under conditions for the cells to be transfected and to produce AAV particles.

The terms “empty capsid” and “empty particle”, refer to an AAV particle that has an AAV protein shell but that lacks in whole or part a nucleic acid that encodes a protein or is transcribed into a transcript of interest flanked by AAV ITRs, i.e. a vector. Accordingly, the empty capsid does not function to transfer a nucleic acid that encodes a protein or is transcribed into a transcript of interest into the host cell.

The term “endogenous” denotes that something is naturally occurring within a cell; naturally produced by a cell; likewise, an “endogenous gene locus/cell-endogenous gene locus” is a naturally occurring locus in a cell.

As used herein, the term “exogenous” indicates that a nucleotide sequence does not originate from a specific cell and is introduced into said cell by DNA delivery methods, e.g., by transfection, electroporation, or transduction by viral vectors. Thus, an exogenous nucleotide sequence is an artificial sequence wherein the artificiality can originate, e.g., from the combination of subsequences of different origin (e.g. a combination of a recombinase recognition sequence with an SV40 promoter and a coding sequence of green fluorescent protein is an artificial nucleic acid) or from the deletion of parts of a sequence (e.g. a sequence coding only the extracellular domain of a membrane-bound receptor or a cDNA) or the mutation of nucleobases. The term “endogenous” refers to a nucleotide sequence originating from a cell. An “exogenous” nucleotide sequence can have an “endogenous” counterpart that is identical in base compositions, but where the sequence is becoming an “exogenous” sequence by its introduction into the cell, e.g., via recombinant DNA technology.

The term “fed-batch cell culture,” as used herein refers to a culture wherein the cells and culture medium are supplied to the culturing bioreactor initially, and additional culture nutrients are fed, continuously or in discrete increments, to the culture during the culturing process, with or without periodic cell and/or product harvest before termination of culture.

An “isolated” composition is one, which has been separated from one or more component(s) of its natural environment. In some embodiments, a composition is purified to greater than 95% or 99% purity as determined by, for example, electrophoretic (e.g., SDS-PAGE, isoelectric focusing (IEF), capillary electrophoresis, CE-SDS) or chromatographic (e.g., size exclusion chromatography or ion exchange or reverse phase HPLC). For review of methods for assessment of e.g. antibody purity, see, e.g., Flatman, S. et al., J. Chrom. B 848 (2007) 79-87.

An “isolated” nucleic acid refers to a nucleic acid molecule that has been separated from one or more component(s) of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated” polypeptide or antibody refers to a polypeptide molecule or antibody molecule that has been separated from one or more component(s) of its natural environment.

The term “mammalian cell comprising an exogenous nucleotide sequence” encompasses cells into which one or more exogenous nucleic acid(s) have been introduced, including the progeny of such cells. These can be the starting point for further genetic modification. Thus, the term “a mammalian cell comprising an exogenous nucleotide sequence” encompasses a cell comprising an exogenous nucleotide sequence integrated at a single site within a locus of the genome of said mammalian cell, wherein the exogenous nucleotide sequence comprises at least a first and a second recombination recognition site (these recombination recognition sites are different) flanking at least one first selection marker. In certain embodiments, the mammalian cell comprising an exogenous nucleotide sequence is a cell comprising an exogenous nucleotide sequence integrated at a single site within a locus of the genome of said cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

A “mammalian cell comprising an exogenous nucleotide sequence” and a “recombinant cell” are both “transfected cells”. This term includes the primary transfected cell as well as progeny derived therefrom without regard to the number of passages. Progeny may, e.g., not be completely identical in nucleic acid content to a parent cell, but may contain mutations. Mutant progeny that has the same function or biological activity as in the originally transfected cell are encompassed.

The “nucleic acids encoding AAV packaging proteins” refer generally to one or more nucleic acid molecule(s) that includes nucleotide sequences providing AAV functions deleted from an AAV vector, which is(are) to be used to produce a transduction competent recombinant AAV particle. The nucleic acids encoding AAV packaging proteins are commonly used to provide expression of AAV rep and/or cap genes to complement missing AAV functions that are necessary for AAV replication; however, the nucleic acid constructs lack AAV ITRs and can neither replicate nor package themselves. Nucleic acids encoding AAV packaging proteins can be in the form of a plasmid, phage, transposon, cosmid, virus, or particle. A number of nucleic acid constructs have been described, such as the commonly used plasmids pAAV/Ad and pIM29+45, which encode both rep and cap gene expression products. See, e.g., Samulski et al. (1989) J. Virol. 63:3822-3828; and McCarty et al. (1991) J. Virol. 65:2936-2945. A number of plasmids have been described which encode rep and/or cap gene expression products (e.g., U.S. Pat. Nos. 5,139,941 and 6,376,237). Any one of these nucleic acids encoding AAV packaging proteins can comprise the DNA element or nucleic acid according to the invention.

The term “nucleic acids encoding helper proteins” refers generally to one or more nucleic acid molecule(s) that include nucleotide sequences encoding proteins and/or RNA molecules that provide adenoviral helper function(s). A plasmid with nucleic acid(s) encoding helper protein(s) can be transfected into a suitable cell, wherein the plasmid is then capable of supporting AAV particle production in said cell. Any one of these nucleic acids encoding helper proteins can comprise the DNA element or nucleic acid according to the invention. Expressly excluded from the term are infectious viral particles, as they exist in nature, such as adenovirus, herpesvirus or vaccinia virus particles.

As used herein, the term “operably linked” refers to a juxtaposition of two or more components, wherein the components are in a relationship permitting them to function in their intended manner. For example, a promoter and/or an enhancer is operably linked to a coding sequence/open reading frame/gene if the promoter and/or enhancer acts to modulate the transcription of the coding sequence/open reading frame/gene. In certain embodiments, DNA sequences that are “operably linked” are contiguous. In certain embodiments, e.g., when it is necessary to join two protein encoding regions, such as a secretory leader and a polypeptide, the sequences are contiguous and in the same reading frame. In certain embodiments, an operably linked promoter is located upstream of the coding sequence/open reading frame/gene and can be adjacent to it. In certain embodiments, e.g., with respect to enhancer sequences modulating the expression of a coding sequence/open reading frame/gene, the two components can be operably linked although not adjacent. An enhancer is operably linked to a coding sequence/open reading frame/gene if the enhancer increases transcription of the coding sequence/open reading frame/gene. Operably linked enhancers can be located upstream, within, or downstream of coding sequences/open reading frames/genes and can be located at a considerable distance from the promoter of the coding sequence/open reading frame/gene.

The term “packaging proteins” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-I) and vaccinia virus.

As used herein, “AAV packaging proteins” refer to AAV-derived sequences, which function in trans for productive AAV replication. Thus, AAV packaging proteins are encoded by the major AAV open reading frames (ORFs), rep and cap. The rep proteins have been shown to possess many functions, including, among others: recognition, binding and nicking of the AAV origin of DNA replication; DNA helicase activity; and modulation of transcription from AAV (or other heterologous) promoters. The cap (capsid) proteins supply necessary packaging functions. AAV packaging proteins are used herein to complement AAV functions in trans that are missing from AAV vectors.

As used herein, “perfusion” or “perfusion culture”, also sometimes referred to as continuous culture, refers to a culture by which the cells are restrained in the culture by, e.g., filtration, encapsulation, anchoring to micro carriers, etc., and the culture medium is continuously, step-wise or intermittently introduced (or any combination of these) and removed from the culturing bioreactor.

The terms “propagate” and “pre-culture” are herein used interchangeably and refer to the step of increasing the number of cells in a cell culture, starting with the inoculation of fresh cultivation medium with an aliquot of cells and maintaining culture conditions for the cells to grow, in one preferred embodiment exponentially, and divide until a desired cell density, i.e. a first pre-determined cell density, is achieved. Generally, cells can be propagated using different methods, such as batch, fed-batch or perfusion culture. The term “propagate” includes the splitting of the cells during propagating, i.e. the decrease of the number of cells (cell density) by removing an aliquot of cultivation medium containing cells and replacing it with/adding a defined aliquot of fresh cultivation medium.

The term “proteinaceous compound” as used herein denotes a heteromultimeric molecule comprising at least one polypeptide, which has been produced in functional form in a mammalian cell. Exemplary proteinaceous compounds are adeno-associated virus particles (AAV particles) comprising a capsid formed of capsid polypeptides and a single stranded DNA molecule, which is a non-polypeptide component.

The term “recombinant cell” as used herein denotes a cell after final genetic modification, such as, e.g., a cell expressing a polypeptide of interest or producing a rAAV particle of interest and that can be used for the production of said polypeptide of interest or rAAV particle of interest at any scale. For example, “a mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”. Although the cell is still capable of performing further RMCE reactions, it is not intended to do so.

A “recombinant AAV vector” is derived from the wild-type genome of a virus, such as AAV by using molecular biological methods to remove the wild type genome from the virus (e.g., AAV), and replacing it with a non-native nucleic acid, such as a nucleic acid transcribed into a transcript or that encodes a protein. Typically, for AAV one or both inverted terminal repeat (ITR) sequences of the wild-type AAV genome are retained in the recombinant AAV vector. A “recombinant” AAV vector is distinguished from a wild-type viral AAV genome, since all or a part of the viral genome has been replaced with a non-native (i.e., heterologous) sequence with respect to the viral genomic nucleic acid. Incorporation of a non-native sequence therefore defines the viral vector (e.g., AAV) as a “recombinant” vector, which in the case of AAV can be referred to as a “rAAV vector.”

A recombinant vector (e.g., AAV) sequence can be packaged—referred to herein as a “particle” for subsequent infection (transduction) of a cell, ex vivo, in vitro or in vivo. Where a recombinant vector sequence is encapsulated or packaged into an AAV particle, the particle can also be referred to as a “rAAV”. Such particles include proteins that encapsulate or package the vector genome. Particular examples include viral envelope proteins, and in the case of AAV, capsid proteins, such as AAV VP1, VP2 and VP3.

As used herein, the term “selection marker” denotes a gene that allows cells carrying the gene to be specifically selected for or against, in the presence of a corresponding selection agent. For example, but not by way of limitation, a selection marker can allow the host cell transformed with the selection marker gene to be positively selected for in the presence of the respective selection agent (selective cultivation conditions); a non-transformed host cell would not be capable of growing or surviving under the selective cultivation conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively be a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells expressing such a molecule can be distinguished from cells not harboring this gene, e.g., by the detection or absence, respectively, of the fluorescence emitted by the encoded polypeptide.

As used herein, the term “serotype” is a distinction based on AAV capsids being serologically distinct. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

The terms “transduce” and “transfect” refer to introduction of a molecule such as a nucleic acid (viral vector, plasmid) into a cell. A cell has been “transduced” or “transfected” when exogenous nucleic acid has been introduced inside the cell membrane. Accordingly, a “transduced cell” is a cell into which a “nucleic acid” or “polynucleotide” has been introduced, or a progeny thereof in which an exogenous nucleic acid has been introduced. In particular embodiments, a “transduced” cell (e.g., in a mammal, such as a cell or tissue or organ cell) has a genetic change following incorporation of an exogenous molecule, for example, a nucleic acid (e.g., a transgene). A “transduced” cell(s) can be propagated and the introduced nucleic acid transcribed and/or protein expressed.

In a “transduced” or “transfected” cell, the nucleic acid (viral vector, plasmid) may or may not be integrated into genomic nucleic acid. If an introduced nucleic acid becomes integrated into the nucleic acid (genomic DNA) of the recipient cell or organism, it can be stably maintained in that cell or organism and further passed on to or inherited by progeny cells or organisms of the recipient cell or organism. Finally, the introduced nucleic acid may exist in the recipient cell or host organism extrachromosomally, or only transiently. A number of techniques are known, see, e.g., Graham et al. (1973) Virology, 52:456; Sambrook et al. (1989) Molecular Cloning, a laboratory manual, Cold Spring Harbor Laboratories, New York; Davis et al. (1986) Basic Methods in Molecular Biology, Elsevier; and Chu et al. (1981) Gene 13:197. Such techniques can be used to introduce one or more exogenous DNA moieties into suitable host cells.

The term “transgene” is used herein to conveniently refer to a nucleic acid that is intended or has been introduced into a cell or organism. Transgenes include any nucleic acid, such as a gene that is transcribed into a transcript or that encodes a polypeptide or protein.

A “vector” refers to the portion of the recombinant plasmid sequence ultimately packaged or encapsulated, either directly or in form of a single strand or RNA, to form a viral (e.g., AAV) particle. In cases recombinant plasmids are used to construct or manufacture recombinant viral particles, the viral particle does not include the portion of the “plasmid” that does not correspond to the vector sequence of the recombinant plasmid. This non-vector portion of the recombinant plasmid is referred to as the “plasmid backbone”, which is important for cloning and amplification of the plasmid, a process that is needed for propagation and recombinant virus production, but is not itself packaged or encapsulated into virus (e.g., AAV) particles. Thus, a “vector” refers to the nucleic acid that is packaged or encapsulated by a virus particle (e.g., AAV).

### Recombinant Cell Line Generation

Generally, for efficient as well as large-scale production of a proteinaceous compound of interest, such as e.g. a rAAV particle or a therapeutic polypeptide, a cell expressing and, if possible, also secreting said proteinaceous compound is required. Such a cell is termed “recombinant cell” or “recombinant production cell”.

In a first step, a suitable host cell is transfected with the required nucleic acid sequences encoding said proteinaceous compound of interest. Transfection of additional helper polypeptides may be necessary.

For expression of a coding sequence, i.e. of an open reading frame, additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case the proteinaceous compound of interest is an AAV particle, which is composed of different (monomeric) polypeptides and a single stranded DNA molecule and which in addition requires other co-factors for production and encapsulation, a multitude of expression cassettes differing in the contained open reading frames/coding sequences are required. In this case, at least an expression cassette for each of the transgene, the different polypeptides forming the capsid of the AAV vector, for the required helper functions as well as the VA RNA are required. Thus, individual expression cassettes for each of the helper E1A, E1B, E2A, E4orf6, the VA RNA, the rep and cap genes are required.

As outlined in the previous paragraphs, the more complex the proteinaceous compound of interest or the higher the number of additional required helper polypeptides and/or RNAs, respectively, the higher is the number of required, different expression cassettes. Inherently with the number of expression cassettes, also the size of the nucleic acid to be integrated into the genome of the host cell increases. However, there is a practical upper limit to the size of a nucleic acid that can be transferred, which is in the range of about 15 kbps (kilo-base-pairs). Above this limit handling and processing efficiency profoundly drops. This issue can be addressed by using two or more separate nucleic acids. Thereby the different expression cassettes are allocated to different nucleic acids, whereby each nucleic acid comprises only some of the expression cassettes.

### Recombinant Cell

Generally, for efficient as well as large-scale production of a proteinaceous compound of interest, such as e.g. a rAAV particle or a therapeutic polypeptide, a cell expressing and, if possible, also secreting said proteinaceous compound is required. Such a cell is termed “recombinant cell” or “recombinant production cell”.

For the generation of a “recombinant production cell” a suitable mammalian cell is transfected with the required nucleic acid sequences encoding said proteinaceous compound of interest. Transfection of additional helper polypeptides may be necessary.

For the generation of stable recombinant production cells, a second step follows, wherein a single cell stably expressing the proteinaceous compound of interest is selected. This can be done, e.g., based on the co-expression of a selection marker, which had been co-transfected with the nucleic acid sequences encoding the proteinaceous compound of interest, or be the expression of the proteinaceous compound itself.

For expression of a coding sequence, i.e. of an open reading frame, additional regulatory elements, such as a promoter and polyadenylation signal (sequence), are necessary. Thus, an open reading frame is operably linked to said additional regulatory elements for transcription. This can be achieved by integrating it into a so-called expression cassette. The minimal regulatory elements required for an expression cassette to be functional in a mammalian cell are a promoter functional in said mammalian cell, which is located upstream, i.e. 5′, to the open reading frame, and a polyadenylation signal (sequence) functional in said mammalian cell, which is located downstream, i.e. 3′, to the open reading frame. Additionally a terminator sequence may be present 3′ to the polyadenylation signal (sequence). For expression, the promoter, the open reading frame/coding region and the polyadenylation signal sequence have to be arranged in an operably linked form.

Likewise, a nucleic acid that is transcribed into a non-protein coding RNA is called “RNA gene”. Also for expression of an RNA gene, additional regulatory elements, such as a promoter and a transcription termination signal or polyadenylation signal (sequence), are necessary. The nature and localization of such elements depends on the RNA polymerase that is intended to drive the expression of the RNA gene. Thus, an RNA gene is normally also integrated into an expression cassette.

In case the proteinaceous compound of interest is an AAV particle, which is composed of different (monomeric) capsid polypeptides and a single stranded DNA molecule and which in addition requires other adenoviral helper functions for production and encapsulation, a multitude of expression cassettes differing in the contained open reading frames/coding sequences are required. In this case, at least an expression cassette for each of the transgene, the different polypeptides forming the capsid of the AAV vector, for the required helper functions as well as the VA RNA are required. Thus, individual expression cassettes for each of the helper E1A, E1B, E2A, E4orf6, the VA RNA, the rep and cap genes are required.

As outlined in the previous paragraphs, the more complex the proteinaceous compound of interest or the higher the number of additional required helper polypeptides and/or RNAs, respectively, the higher is the number of required, different expression cassettes. Inherently with the number of expression cassettes, also the total size of the nucleic acid. However, there is a practical upper limit to the size of a nucleic acid that can be transferred, which is in the range of about 15 kbps (kilo-base-pairs). Above this limit handling and processing efficiency profoundly drops. This issue can be addressed by using two or more separate plasmids. Thereby the different expression cassettes are allocated to different plasmids, whereby each plasmid comprises only some of the expression cassettes.

For stable cell line development random integration (RI) of the nucleic acid(s) carrying the expression cassettes for the proteinaceous compound of interest can be used. In general, by using RI the nucleic acids or fragments thereof integrate into the host cell's genome at random.

Alternatively, to RI, targeted integration (TI) can be used for CLD. In TI CLD, one or more nucleic acid(s) comprising the different expression cassettes is/are introduced at a predetermined locus in the host cell's genome.

In TI either homologous recombination or a recombinase mediated cassette exchange reaction (RMCE) can be employed for the integration of the nucleic acid (a) comprising the respective expression cassettes into the specific locus in the genome of the TI host cell.

In certain embodiments of all aspects and embodiments, each of the expression cassettes comprise in 5′-to-3′ direction a promoter, an open reading frame/coding sequence or an RNA gene and a polyadenylation signal sequence, and/or a terminator sequence. In certain embodiments, the open reading frame encodes a polypeptide and the expression cassette comprises a polyadenylation signal sequence with or without additional terminator sequence. In certain embodiments, the expression cassette comprises a RNA gene, the promoter is a type 2 Pol III promoter and a polyadenylation signal sequence or a polyU terminator is present. See, e.g., Song et al. Biochemical and Biophysical Research Communications 323 (2004) 573-578. In certain embodiments, the expression cassette comprises a RNA gene, the promoter is a type 2 Pol III promoter and a polyU terminator sequence.

In certain embodiments of all aspects and embodiments, the open reading frame encodes a polypeptide, the promoter is the human CMV promoter with or without intron A, the polyadenylation signal sequence is the bGH (bovine growth hormone) polyA signal sequence and the terminator is the hGT (human gastrin terminator).

In certain embodiments of all aspects and embodiments the promoter is the human CMV promoter with intron A, the polyadenylation signal sequence is the bGH polyadenylation signal sequence and the terminator is the hGT, except for the expression cassette of the RNA gene and the expression cassette of the selection marker, wherein for the selection marker the promoter is the SV40 promoter and the polyadenylation signal sequence is the SV40 polyadenylation signal sequence and a terminator is absent, and wherein for the RNA gene the promoter is a wild-type type 2 polymerase III promoter and the terminator is a polymerase II or III terminator.

It has to be pointed out that in certain embodiments the current invention does not encompass permanent human cell lines comprising a nucleic acid sequence for the adenoviral gene functions E1A and E1B and concomitantly the nucleic acid sequence for the SV40 large T-antigen or the Epstein-Barr virus (EBV) nuclear antigen 1 (EBNA-1).

### Adeno-Associated Virus (AAV)

For a general review of AAVs and of the adenovirus or herpes helper functions see, Berns and Bohensky, Advances in Virus Research, Academic Press., 32 (1987) 243-306. The genome of AAV is described in Srivastava et al., J. Virol., 45 (1983) 555-564. In U.S. Pat. No. 4,797,368 design considerations for constructing recombinant AAV vectors are described (see also WO 93/24641). Additional references describing AAV vectors are West et al., Virol. 160 (1987) 38-47; Kotin, Hum. Gene Ther. 5 (1994) 793-801; and Muzyczka J. Clin. Invest. 94 (1994) 1351. Construction of recombinant AAV vectors described in U.S. Pat. No. 5,173,414; Lebkowski et al., Mol. Cell. Biol. 8 (1988) 3988-3996; Tratschin et al., Mol. Cell. Biol. 5 (1985) 3251-3260; Tratschin et al., Mol. Cell. Biol., 4 (1994) 2072-2081; Hermonat and Muzyczka Proc. Natl. Acad. Sci. USA 81 (1984) 6466-6470; Samulski et al. J. Virol. 63 (1989) 3822-3828.

An adeno-associated virus (AAV) is a replication-deficient parvovirus. It can replicate only in cells, in which certain viral functions are provided by a co-infecting helper virus, such as adenoviruses, herpesviruses and, in some cases, poxviruses such as vaccinia. Nevertheless, an AAV can replicate in virtually any cell line of human, simian or rodent origin provided that the appropriate helper viral functions are present.

Without helper viral genes being present, an AAV establishes latency in its host cell. Its genome integrates into a specific site in chromosome 19 [(Chr) 19 (q13.4)], which is termed the adeno-associated virus integration site 1 (AAVS1). For specific serotypes, such as AAV-2 other integration sites have been found, such as, e.g., on chromosome 5 [(Chr) 5 (p13.3)], termed AAVS2, and on chromosome 3 [(Chr) 3 (p24.3)], termed AAVS3.

AAVs are categorized into different serotypes. These have been allocated based on parameters, such as hemagglutination, tumorigenicity and DNA sequence homology. Up to now, more than 10 different serotypes and more than a hundred sequences corresponding to different clades of AAV have been identified.

The capsid protein type and symmetry determines the tissue tropism of the respective AAV. For example, AAV-2, AAV-4 and AAV-5 are specific to retina, AAV-2, AAV-5, AAV-8, AAV-9 and AAVrh-10 are specific for brain, AAV-1, AAV-2, AAV-6, AAV-8 and AAV-9 are specific for cardiac tissue, AAV-1, AAV-2, AAV-5, AAV-6, AAV-7, AAV-8, AAV-9 and AAV-10 are specific for liver, AAV-1, AAV-2, AAV-5 and AAV-9 are specific for lung.

Pseudotyping denotes a process comprising the cross packaging of the AAV genome between various serotypes, i.e. the genome is packaged with differently originating capsid proteins.

The wild-type AAV genome has a size of about 4.7 kb. The AAV genome further comprises two overlapping genes named rep and cap, which comprise multiple open reading frames (see, e.g., Srivastava et al., J. Viral., 45 (1983) 555-564; Hermonat et al., J. Viral. 51 (1984) 329-339; Tratschin et al., J. Virol., 51 (1984) 611-619). The Rep protein encoding open reading frame provides for four proteins of different size, which are termed Rep78, Rep68, Rep52 and Rep40. These are involved in replication, rescue and integration of the AAV. The Cap protein encoding open reading frame provides four proteins, which are termed VP1, VP2, VP3, and AAP. VP1, VP2 and VP3 are part of the proteinaceous capsid of the AAV particles. The combined rep and cap open reading frames are flanked at their 5′- and 3′-ends by so-called inverted terminal repeats (ITRs). For replication, an AAV requires in addition to the Rep and Cap proteins the products of the genes E1A, E1B, E4orf6, E2A and VA of an adenovirus or corresponding factors of another helper virus.

In the case of an AAV of the serotype 2 (AAV-2), for example, the ITRs each have a length of 145 nucleotides and flank a coding sequence region of about 4470 nucleotides. Of the ITR's 145 nucleotides 125 nucleotides have a palindromic sequence and can form a T-shaped hairpin structure. This structure has the function of a primer during viral replication. The remaining 20, non-paired, nucleotides are denoted as D-sequence.

The AAV genome harbors three transcription promoters P5, P19, and P40 (Laughlin et al., Proc. Natl. Acad. Sci. USA 76 (1979) 5567-5571) for the expression of the rep and cap genes.

The ITR sequences have to be present in cis to the coding region. The ITRs provide a functional origin of replication (ori), signals required for integration into the target cell's genome, and efficient excision and rescue from host cell chromosomes or recombinant plasmids. The ITRs further comprise origin of replication like-elements, such as a Rep-protein binding site (RBS) and a terminal resolution site (TRS). It has been found that the ITRs themselves can have the function of a transcription promoter in an AAV vector (Flotte et al., J. Biol. Chem. 268 (1993) 3781-3790; Flotte et al., Proc. Natl. Acad. Sci. USA 93 (1993) 10163-10167).

For replication and encapsidation, respectively, of the viral single-stranded DNA genome an in trans organization of the rep and cap gene products are required.

The rep gene locus comprises two internal promoters, termed P5 and P19. It comprises open reading frames for four proteins. Promoter P5 is operably linked to a nucleic acid sequence providing for non-spliced 4.2 kb mRNA encoding the Rep protein Rep78 (chromatin nickase to arrest cell cycle), and a spliced 3.9 kb mRNA encoding the Rep protein Rep68 (site-specific endonuclease). Promoter P19 is operably linked to a nucleic acid sequence providing for a non-spliced mRNA encoding the Rep protein Rep52 and a spliced 3.3 kb mRNA encoding the Rep protein Rep40 (DNA helicases for accumulation and packaging).

The two larger Rep proteins, Rep78 and Rep68, are essential for AAV duplex DNA replication, whereas the smaller Rep proteins, Rep52 and Rep40, seem to be essential for progeny, single-strand DNA accumulation (Chejanovsky & Carter, Virology 173 (1989) 120-128).

The larger Rep proteins, Rep68 and Rep78, can specifically bind to the hairpin conformation of the AAV ITR. They exhibit defined enzyme activities, which are required for resolving replication at the AAV termini. Expression of Rep78 or Rep68 could be sufficient for infectious particle formation (Holscher, C., et al. J. Virol. 68 (1994) 7169-7177 and 69 (1995) 6880-6885).

It is deemed that all Rep proteins, primarily Rep78 and Rep68, exhibit regulatory activities, such as induction and suppression of AAV genes as well as inhibitory effects on cell growth (Tratschin et al., Mol. Cell. Biol. 6 (1986) 2884-2894; Labow et al., Mol. Cell. Biol., 7 (1987) 1320-1325; Khleif et al., Virology, 181 (1991) 738-741).

Recombinant overexpression of Rep78 results in phenotype with reduced cell growth due to the induction of DNA damage. Thereby the host cell is arrested in the S phase, whereby latent infection by the virus is facilitated (Berthet, C., et al., Proc. Natl. Acad. Sci. USA 102 (2005) 13634-13639).

Tratschin et al. reported that the P5 promoter is negatively auto-regulated by Rep78 or Rep68 (Tratschin et al., Mol. Cell. Biol. 6 (1986) 2884-2894). Due to the toxic effects of expression of the Rep protein, only very low expression has been reported for certain cell lines after stable integration of AAV (see, e.g., Mendelson et al., Virol. 166 (1988) 154-165).

The cap gene locus comprises one promoter, termed P40. Promoter P40 is operably linked to a nucleic acid sequence providing for 2.6 kb mRNA, which, by alternative splicing and use of alternative start codons, encodes the Cap proteins VP1 (87 kDa, non-spliced mRNA transcript), VP2 (72 kDa, from the spliced mRNA transcript), and VP3 (61 kDa, from alternative start codon). VP1 to VP3 constitute the building blocks of the viral capsid. The capsid has the function to bind to a cell surface receptor and allow for intracellular trafficking of the virus. VP3 accounts for about 90% of total viral particle protein. Nevertheless, all three proteins are essential for effective capsid production.

It has been reported that inactivation of all three capsid proteins VP1 to VP3 prevents accumulation of single-strand progeny AAV DNA. Mutations in the VP1 amino-terminus (“Lip-negative” or “Inf-negative”) still allows for assembly of single-stranded DNA into viral particles whereby the infectious titer is greatly reduced.

The AAP open reading frame is encoding the assembly activating protein (AAP). It has a size of about 22 kDa and transports the native VP proteins into the nucleolar region for capsid assembly. This open reading frame is located upstream of the VP3 protein encoding sequence.

In individual AAV particles, only one single-stranded DNA molecule is contained. This may be either the “plus” or “minus” strand. AAV viral particles containing a DNA molecule are infectious. Inside the infected cell, the parental infecting single strand is converted into a double strand, which is subsequently amplified. The amplification results in a large pool of double stranded DNA molecules from which single strands are displaced and packaged into capsids.

Adeno-associated viral (AAV) vectors can transduce dividing cells as well as resting cells. It can be assumed that a transgene introduced using an AAV vector into a target cell will be expressed for a long period. One drawback of using an AAV vector is the limitation of the size of the transgene that can be introduced into cells.

Viral vectors such as parvo-virus particles, including AAV serotypes and variants thereof, provide a means for delivery of nucleic acid into cells ex vivo, in vitro and in vivo, which encode proteins such that the cells express the encoded protein. AAVs are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, AAV vectors are able to deliver heterologous polynucleotide sequences (e.g., therapeutic proteins and agents) to human patients without causing substantial AAV pathogenesis or disease.

Viral vectors, which may be used, include, but are not limited to, adeno-associated virus (AAV) particles of multiple serotypes (e.g., AAV-1 to AAV-12, and others) and hybrid/chimeric AAV particles.

AAV particles may be used to advantage as vehicles for effective gene delivery. Such particles possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

Recombinant AAV particles do not typically include viral genes associated with pathogenesis. Such vectors typically have one or more of the wild-type AAV genes deleted in whole or in part, for example, rep and/or cap genes, but retain at least one functional flanking ITR sequence, as necessary for the rescue, replication, and packaging of the recombinant vector into an AAV particle. For example, only the essential parts of the vector e.g., the ITR and LTR elements, respectively, are included. An AAV vector genome would therefore include sequences required in cis for replication and packaging (e.g., functional ITR sequences).

Recombinant AAV vectors, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, 2i8, AAV rh74 or AAV 7m8 for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be an AAV1, AAV3, AAV4, AAV5, AAV6, AAV7, AAV8, AAV9, AAV10, AAV11, AAV12, AAV-2i8, AAV rh74, AAV 7m8 or a variant thereof, for example. AAV variants include variants and chimeras of AAV1, AAV2, AAV3, AAV4, AAV5, AAV6, AAV7, AAV8, AAV9, AAV10, AAV11, AAV12, AAV-2i8, AAV rh74 and AAV 7m8 capsids.

In certain embodiments of all aspects and embodiments, adeno-associated virus (AAV) vectors include AAV1, AAV2, AAV3, AAV4, AAV5, AAV6, AAV7, AAV8, AAV9, AAV10, AAV11, AAV12, AAV-2i8, AAV rh74 and AAV 7m8, as well as variants (e.g., capsid variants, such as amino acid insertions, additions, substitutions and deletions) thereof, for example, as set forth in WO 2013/158879, WO 2015/013313 and US 2013/0059732 (disclosing LK01, LK02, LK03, etc.).

AAV and AAV variants (e.g., capsid variants) serotypes (e.g., VP1, VP2, and/or VP3 sequences) may or may not be distinct from other AAV serotypes, including, for example, AAV1-AAV12 (e.g., distinct from VP1, VP2, and/or VP3 sequences of any of AAV1-AAV12 serotypes).

In certain embodiments of all aspects and embodiments, an AAV particle related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, AAV-2i8, AAV rh74 or AAV 7m8 (e.g., such as an ITR, or a VP1, VP2, and/or VP3 sequences).

Compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, AAV-2i8, AAV rh74, or AAV 7m8, but are distinct from and not identical to known AAV genes or proteins, such as AAV1, AAV2, AAV3, AAV4, AAV5, AAV6, AAV7, AAV8, AAV9, AAV10, AAV11, AAV12, AAV-2i8, AAV rh74, or AAV 7m8, genes or proteins, etc. In certain embodiments of all aspects and embodiments, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, AAV-2i8, AAV rh74, or AAV 7m8 (e.g., VP1, VP2 and/or VP3 capsid or ITR). In certain embodiments, an AAV variant has 1, 2, 3, 4, 5, 5-10, 10-15, 15-20 or more amino acid substitutions.

Recombinant AAV particles, including AAV1, AAV2, AAV3, AAV4, AAV5, AAV6, AAV7, AAV8, AAV9, AAV10, AAV11, AAV12, AAV-2i8, AAV rh74 or AAV 7m8, and variant, related, hybrid and chimeric sequences, can be constructed using recombinant techniques that are known to the skilled artisan, to include one or more nucleic acid sequences (transgenes) flanked with one or more functional AAV ITR sequences.

Recombinant particles (e.g., rAAV particles) can be incorporated into pharmaceutical compositions. Such pharmaceutical compositions are useful for, among other things, administration and delivery to a subject in vivo or ex vivo. In certain embodiments, the pharmaceutical composition contains a pharmaceutically acceptable carrier or excipient. Such excipients include any pharmaceutical agent that does not itself induce an immune response harmful to the individual receiving the composition, and which may be administered without undue toxicity.

Protocols for the generation of adenoviral vectors have been described in U.S. Pat. Nos. 5,998,205; 6,228,646; 6,093,699; 6,100,242; WO 94/17810 and WO 94/23744, which are incorporated herein by reference in their entirety.

Recombinant AAV Particles (rAAV Particles)

Different methods that are known in the art for generating rAAV particles. For example, transfection using AAV plasmid and AAV helper sequences in conjunction with co-infection with one AAV helper virus (e.g., adenovirus, herpesvirus, or vaccinia virus) or transfection with a recombinant AAV plasmid, an AAV helper plasmid, and an helper function plasmid. Non-limiting methods for generating rAAV particles are described, for example, in U.S. Pat. Nos. 6,001,650, 6,004,797, WO 2017/096039, and WO 2018/226887. Following recombinant rAAV particle production (i.e. particle generation in cell culture systems), rAAV particles can be obtained from the host cells and cell culture supernatant and purified.

For the generation of recombinant AAV particles, expression of the Rep and Cap proteins, the helper proteins E1A, E1B, E2A and E4orf6 as well as the adenoviral VA RNA in a single mammalian cell is required. The helper proteins E1A, E1B, E2A and E4orf6 can be expressed using any promoter as shown by Matsushita et al. (Gene Ther. 5 (1998) 938-945), especially the CMV IE promoter. Thus, any promoter can be used.

Generally, to produce recombinant AAV particles, different, complementing plasmids are co-transfected into a host cell. One of the plasmids comprises the transgene sandwiched between the two cis acting AAV ITRs. The missing AAV elements required for replication and subsequent packaging of progeny recombinant genomes, i.e. the open reading frames for the Rep and Cap proteins, are contained in trans on a second plasmid. The overexpression of the Rep proteins results in inhibitory effects on cell growth (Li, J., et al., J. Virol. 71 (1997) 5236-5243). Additionally, a third plasmid comprising the genes of a helper virus, i.e. E1, E4orf6, E2A and VA from adenovirus, is required for AAV replication.

To reduce the number of required plasmids, Rep, Cap and the adenovirus helper genes may be combined on a single plasmid.

Alternatively, the host cell may already stably express the E1 gene products. Such a cell is a HEK293 cell. The human embryonic kidney clone denoted as 293 was generated back in 1977 by integrating adenoviral DNA into human embryonic kidney cells (HEK cells) (Graham, F. L., et al., J. Gen. Virol. 36 (1977) 59-74). The HEK293 cell line comprises base pair 1 to 4344 of the adenovirus serotype 5 genome. This encompasses the E1A and E1B genes as well as the adenoviral packaging signals (Louis, N., et al., Virology 233 (1997) 423-429).

When using HEK293 cells the missing E2A, E4orf6 and VA genes can be introduced either by co-infection with an adenovirus or by co-transfection with an E2A-, E4orf6- and VA-expressing plasmid (see, e.g., Samulski, R. J., et al., J. Virol. 63 (1989) 3822-3828; Allen, J. M., et al., J. Virol. 71 (1997) 6816-6822; Tamayose, K., et al., Hum. Gene Ther. 7 (1996) 507-513; Flotte, T. R., et al., Gene Ther. 2 (1995) 29-37; Conway, J. E., et al., J. Virol. 71 (1997) 8780-8789; Chiorini, J. A., et al., Hum. Gene Ther. 6 (1995) 1531-1541; Ferrari, F. K., et al., J. Virol. 70 (1996) 3227-3234; Salvetti, A., et al., Hum. Gene Ther. 9 (1998) 695-706; Xiao, X., et al., J. Virol. 72 (1998) 2224-2232; Grimm, D., et al., Hum. Gene Ther. 9 (1998) 2745-2760; Zhang, X., et al., Hum. Gene Ther. 10 (1999) 2527-2537). Alternatively, adenovirus/AAV or herpes simplex virus/AAV hybrid vectors can be used (see, e.g., Conway, J. E., et al., J. Virol. 71 (1997) 8780-8789; Johnston, K. M., et al., Hum. Gene Ther. 8 (1997) 359-370; Thrasher, A. J., et al., Gene Ther. 2 (1995) 481-485; Fisher, J. K., et al., Hum. Gene Ther. 7 (1996) 2079-2087; Johnston, K. M., et al., Hum. Gene Ther. 8 (1997) 359-370).

Thus, cell lines in which the rep gene is integrated and expressed tend to grow slowly or express Rep proteins at very low levels.

In order to limit the transgene activity to specific tissues, i.e. to limit the site of integration the transgene can be operably linked to an inducible or tissue specific promoter (see, e.g., Yang, Y., et al. Hum. Gene. Ther. 6 (1995) 1203-1213).

One difficulty in the production of rAAV particles is the inefficient packaging of the rAAV vector, resulting in low titers. Packaging has been difficult for several reasons including
preferred encapsidation of wild-type AAV genomes if they are present;
difficulty in generating sufficient complementing functions such as those provided by the wild-type rep and cap genes due to the inhibitory effect associated with the rep gene products;
the limited efficiency of the co-transfection of the plasmid constructs.

All this is based on the biological properties of the Rep proteins. Especially the inhibitory (cytostatic and cytotoxic) properties of the Rep proteins as well as the ability to reverse the immortalized phenotype of cultured cells is problematic. Additionally, Rep proteins down-regulate their own expression when the widely used AAV P5 promoter is employed (see, e.g., Tratschin et al., Mol. Cell. Biol. 6 (1986) 2884-2894).

In certain embodiments of all aspects and embodiments, the rAAV particles are derived from an AAV selected from the group consisting of AAV1, AAV2, AAV3, AAV4, AAV5, AAV6, AAV7, AAV8, AAV9, AAV10, Rh10, Rh74 and 7m8.

In certain embodiments of all aspects and embodiments, the rAAV particles comprise a capsid sequence having 70% or more sequence identity to an AAV1, AAV2, AAV3, AAV4, AAV5, AAV6, AAV7, AAV8, AAV9, AAV10, Rh10, Rh74, or 7m8 capsid sequence.

In certain embodiments of all aspects and embodiments, the rAAV particles comprise an ITR sequence having 70% or more sequence identity to an AAV1, AAV2, AAV3, AAV4, AAV5, AAV6, AAV7, AAV8, AAV9, or AAV10 ITR sequence.

### E1A E1B E2 and E4

The coding sequences of E1A and E1B (open reading frames) can be derived from a human adenovirus, such as, e.g., in particular of human adenovirus serotype 2 or serotype 5. An exemplary sequence of human Ad5 (adenovirus serotype 5) is found in GenBank entries X02996, AC_000008 and that of an exemplary human Ad2 in GenBank entry AC_000007. Nucleotides 505 to 3522 comprise the nucleic acid sequences encoding E1A and E1B of human adenovirus serotype 5. Plasmid pSTK146 as reported in EP 1 230 354, as well as plasmids pGS119 and pGS122 as reported in WO 2007/056994, can also be used a source for the E1A and E1B open reading frames.

E1A is the first viral helper gene that is expressed after adenoviral DNA enters the cell nucleus. The E1A gene encodes the 12S and 13S proteins, which are based on the same E1A mRNA by alternative splicing. Expression of the 12S and 13S proteins results in the activation of the other viral functions E1B, E2, E3 and E4. Additionally, expression of the 12S and 13S proteins force the cell into the S phase of the cell cycle. If only the E1A-derived proteins are expressed, the cell will dye (apoptosis).

E1B is the second viral helper gene that is expressed. It is activated by the E1A-derived proteins 12S and 13S. The E1B gene derived mRNA can be spliced in two different ways resulting in a first 55 kDa transcript and a second 19 kDa transcript. The E1B 55 kDa protein is involved in the modulation of the cell cycle, the prevention of the transport of cellular mRNA in the late phase of the infection, and the prevention of E1A-induced apoptosis. The E1B 19 kDa protein is involved in the prevention of E1A-induced apoptosis of cells.

The E2 gene encodes different proteins. The E2A transcript codes for the single strand-binding protein (SSBP), which is essential for AAV replication

Also, the E4 gene encodes several proteins. The E4 gene derived 34 kDa protein (E4orf6) prevents the accumulation of cellular mRNAs in the cytoplasm together with the E1B 55 kDa protein, but also promotes the transport of viral RNAs from the cell nucleus into the cytoplasm.

### Adenoviral VA RNA Gene

The viral associated RNA (VA RNA) is a non-coding RNA of adenovirus (Ad), regulating translation. The adenoviral genome comprises two independent copies: VAI (VA RNAI) and VAII (VA RNAII). Both are transcribed by RNA polymerase III (see, e.g., Machitani, M., et al., J. Contr. Rel. 154 (2011) 285-289) from a type 2 polymerases III promoter. For recombinant production, the adenoviral VA RNA gene can be driven by any promoter.

The structure, function, and evolution of adenovirus-associated RNA using a phylogenetic approach was investigated by Ma, Y. and Mathews, M. B. (J. Virol. 70 (1996) 5083-5099). They provided alignments as well as consensus VA RNA sequences based on 47 known human adenovirus serotypes. Said disclosure is herewith incorporated by reference in its entirety into the current application.

VA RNAs, VAI and VAII, are consisting of 157-160 nucleotides (nt).

Depending on the serotype, adenoviruses contain one or two VA RNA genes. VA RNAI is believed to play the dominant pro-viral role, while VA RNAII can partially compensate for the absence of VA RNAI (Vachon, V. K. and Conn, G. L., Virus Res. 212 (2016) 39-52).

The VA RNAs are not essential, but play an important role in efficient viral growth by overcoming cellular antiviral machinery. That is, although VA RNAs are not essential for viral growth, VA RNA-deleted adenovirus cannot grow during the initial step of vector generation, where only a few copies of the viral genome are present per cell, possibly because viral genes other than VA RNAs that block the cellular antiviral machinery may not be sufficiently expressed (see Maekawa, A., et al. Nature Sci. Rep. 3 (2013) 1136).

Maekawa, A., et al. (Nature Sci. Rep. 3 (2013) 1136) reported efficient production of adenovirus vector lacking genes of virus-associated RNAs that disturb cellular RNAi machinery, wherein HEK293 cells that constitutively and highly express flippase recombinase were infected to obtain VA RNA-deleted adenovirus by FLP recombinase-mediated excision of the VA RNA locus.

The human adenovirus 2 VA RNAI corresponds to nucleotides 10586-10810 of GenBank entry AC_000007 sequence. The human adenovirus 5 VA RNAI corresponds to nucleotides 10579-10820 of GenBank entry AC_000008 sequence.

Methods for Producing rAAV Particles

Carter et al. have shown that the entire rep and cap open reading frames in the wild-type AAV genome can be deleted and replaced with a transgene (Carter, B. J., in “Handbook of Parvoviruses”, ed. by P. Tijssen, CRC Press, pp. 155-168 (1990)). Further, it has been reported that the ITRs have to be maintained to retain the function of replication, rescue, packaging, and integration of the transgene into the genome of the target cell.

When cells comprising the respective viral helper genes are transduced by an AAV vector, or, vice versa, when cells comprising an integrated AAV provirus are transduced by a suitable helper virus, then the AAV provirus is activated and enters a lytic infection cycle again (Clark, K. R., et al., Hum. Gene Ther. 6 (1995) 1329-1341; Samulski, R. J., Curr. Opin. Genet. Dev. 3 (1993) 74-80).

Producer cells contain the rep and cap gene sequences, as well as the transgene cassette flanked by ITR sequences on one or more plasmids that are retained via drug selection. Production of rAAV particles in these cell lines generally occurs after their infection with the required helper functions. Therefore, cells are infected either with replication-competent AdV (usually wild type Ad5) or a plasmid comprising the respective helper genes to supply helper virus proteins and initiate rAAV particle production. A packaging cell line differs from a producer cell line as it only contains the rep and cap genes.

Aspects of the current invention are methods of transducing cells with nucleic acids (e.g., plasmids) comprising all required elements for the production of recombinant AAV particles, wherein the cells prior to transfection have been propagated (at least for some time) using perfusion. Thus, as the plasmids encode viral packaging proteins and/or helper proteins the cells can produce recombinant viral particles that include a nucleic acid that encodes a protein of interest or comprises a sequence that is transcribed into a transcript of interest.

The invention provides a viral (e.g., AAV) particle production platform that includes features that distinguish it from current ‘industry-standard’ viral (e.g., AAV) particle production processes by using the method according to the current invention.

More generally, cells transfected or transduced with DNA for the recombinant production of AAV particles can be referred to as “recombinant cell”. Such a cell can be, for example, a yeast cell, an insect cell, or a mammalian cell, and has been used as recipient of a nucleic acid (plasmid) encoding packaging proteins, such as AAV packaging proteins, a nucleic acid (plasmid) encoding helper proteins, and a nucleic acid (plasmid) that encodes a protein or is transcribed into a transcript of interest, i.e. a transgene placed between two AAV ITRs. The term includes the progeny of the original cell, which has been transduced or transfected. It is understood that the progeny of a single parental cell may not necessarily be completely identical in morphology or in genomic or total nucleic acid complement as the original parent, due to natural, accidental, or deliberate mutation.

Numerous cell growth media appropriate for sustaining cell viability or providing cell growth and/or proliferation are commercially available. Examples of such medium include serum free eukaryotic growth mediums, such as medium for sustaining viability or providing for the growth of mammalian (e.g., human) cells. Non-limiting examples include Ham's F12 or F12K medium (Sigma-Aldrich), FreeStyle (FS) F17 medium (Thermo-Fisher Scientific), MEM, DMEM, RPMI-1640 (Thermo-Fisher Scientific) and mixtures thereof. Such media can be supplemented with vitamins and/or trace minerals and/or salts and/or amino acids, such as essential amino acids for mammalian (e.g., human) cells.

Helper protein plasmids can be in the form of a plasmid, phage, transposon or cosmid. In particular, it has been demonstrated that the full-complement of adenovirus genes are not required for helper functions. For example, adenovirus mutants incapable of DNA replication and late gene synthesis have been shown to be permissive for AAV replication. Ito et al., J. Gen. Virol. 9 (1970) 243; Ishibashi et al., Virology 45 (1971) 317.

Mutants within the E2B and E3 regions have been shown to support AAV replication, indicating that the E2B and E3 regions are probably not involved in providing helper function. Carter et al., Virology 126 (1983) 505. However, adenoviruses defective in the E1 region, or having a deleted E4 region, are unable to support AAV replication. Thus, for adenoviral helper proteins, E1A and E4 regions are likely required for AAV replication, either directly or indirectly (see, e.g., Laughlin et al., J. Virol. 41 (1982) 868; Janik et al., Proc. Natl. Acad. Sci. USA 78 (1981) 1925; Carter et al., Virology 126 (1983) 505). Other characterized adenoviral mutants include: E1B (Laughlin et al. (1982), supra; Janik et al. (1981), supra; Ostrove et al., Virology 104 (1980) 502); E2A (Handa et al., J. Gen. Virol. 29 (1975) 239; Strauss et al., J. Virol. 17 (1976) 140; Myers et al., J. Virol. 35 (1980) 665; Jay et al., Proc. Natl. Acad. Sci. USA 78 (1981) 2927; Myers et al., J. Biol. Chem. 256 (1981) 567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)).

Studies of the helper proteins provided by adenoviruses having mutations in the E1B have reported that the E1B 55 kDa protein is required for AAV particle production, while E1B 19 kDa is not. In addition, WO 97/17458 and Matshushita et al. (Gene Therapy 5 (1998) 938-945) described helper function plasmids encoding various adenoviral genes. An example of a helper plasmid comprise an adenovirus VA RNA coding region, an adenovirus E4orf6 coding region, an adenovirus E2A 72 kDa coding region, an adenovirus E1A coding region, and an adenovirus E1B region lacking an intact E1B 55 kDa coding region (see, e.g., WO 01/83797).

Thus, herein is provided a method for producing recombinant AAV vectors or AAV particles comprising said recombinant AAV vectors, which comprise a nucleic acid that encodes a protein or is transcribed into a transcript of interest, using the method according to the current invention for mammalian cell propagation prior to transfection and viral particle production.

For this purpose, three plasmids are co-transfected into a mammalian cell propagated with perfusion according to the current invention. The transgene plasmid (pTransgen) encodes the expression cassette, which is cloned between the AAV ITRs, whereas rep and cap genes are provided in trans by co-transfecting a second packaging plasmid (pRep/Cap) to ensure AAV replication and packaging. The third plasmid, also referred to as helper plasmid (pHelper), contains the minimal helper virus factors, commonly adenoviral E2A, EV and VA genes, but lacking the AAV ITRs.

One aspect of the current invention is a method for producing recombinant AAV vectors or AAV particles comprising said recombinant AAV vectors, which comprise a nucleic acid that encodes a protein or is transcribed into a transcript of interest, comprising the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii) contacting one or more mammalian or insect cells obtained with a method according to the invention with the provided plasmids;
(iv) either further adding a transfection reagent and optionally incubating the plasmid/transfection reagent/cell mixture; or providing a physical means, such as an electric current, to introduce the nucleic acid into the cells;
(v) cultivating the transfected cells;
(vi) harvesting the cultivated cells and/or culture medium from the cultivated cells to produce a cell and/or culture medium harvest; and
(vii) lysing the cells and optionally isolating recombinant AAV vector or AAV particle from the cell and/or culture medium harvest lysate;
thereby producing a recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprising the steps of
(i) providing one or more plasmids comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii) either
   (a) generating a stable transfected cell by contacting one or more mammalian cells obtained with the method according to the invention with the provided plasmids of (i); either further adding a transfection reagent and optionally incubating the plasmid/transfection reagent/cell mixture or providing a physical means, such as an electric current, to introduce the nucleic acid into the cells; selecting a first stably transfected cell; contacting the selected first stably transfected cell with the provided plasmid of (ii); and either further adding a transfection reagent and optionally incubating the plasmid/transfection reagent/cell mixture; or providing a physical means, such as an electric current, to introduce the nucleic acid into the cells; or
   (b) generating a transient transfected cell by contacting one or more mammalian or insect cells obtained with the method according to the invention with the provided plasmids of (i) and (ii); and either further adding a transfection reagent and optionally incubating the plasmid/transfection reagent/cell mixture; or providing a physical means, such as an electric current, to introduce the nucleic acid into the cells;
(iv) cultivating the transfected cell of (iii);
(v) harvesting the cultivated cells and/or culture medium from the cultivated cells to produce a cell and/or culture medium harvest; and
(vi) lysing the cells and optionally isolating recombinant AAV vector or AAV particle from the cell and/or culture medium harvest lysate;
thereby producing recombinant AAV vector or AAV particle comprising a nucleic acid that encodes a protein of interest or is transcribed into a transcript of interest.

One aspect of the current invention is a method for producing recombinant AAV vectors or AAV particles comprising said recombinant AAV vectors, which comprise a nucleic acid that encodes a protein or is transcribed into a transcript of interest, comprises the steps of
(i) providing a mammalian or insect cell comprising nucleic acids encoding AAV packaging proteins and/or nucleic acids encoding helper proteins;
(ii) providing a plasmid comprising a nucleic acid that encodes a protein of interest or is transcribed into a transcript of interest;
(iii) either
   (a) generating a stable transfected cell by contacting one or more mammalian or insect cells obtained with the method according to the invention with the provided plasmids of (i); either further adding a transfection reagent and optionally incubating the plasmid/transfection reagent/cell mixture; or providing a physical means, such as an electric current, to introduce the nucleic acid into the cells; selecting a first stably transfected cell; contacting the selected first stably transfected cell with the provided plasmid of (ii); and either further adding a transfection reagent and optionally incubating the plasmid/transfection reagent/cell mixture; or providing a physical means, such as an electric current, to introduce the nucleic acid into the cells; or
   (b) generating a transient transfected cell by contacting one or more mammalian or insect cells obtained with the method according to the invention with the provided plasmids of (i) and (ii); and either further adding a transfection reagent and optionally incubating the plasmid/transfection reagent/cell mixture; or providing a physical means, such as an electric current, to introduce the nucleic acid into the cells;
(iv) cultivating the transfected cell of (iii);
(v) harvesting the cultivated cells and/or culture medium from the cultivated cells to produce a cell and/or culture medium harvest; and
(vi) lysing the cells and optionally isolating and/or purifying recombinant AAV vector or AAV particle from the cell and/or culture medium harvest;
thereby producing recombinant AAV vector or AAV particle comprising a nucleic acid that encodes a protein of interest or is transcribed into a transcript of interest.

The introduction of the nucleic acid (plasmids) into cells can be done in multiple ways.

Diverse methods for the DNA transfer into mammalian cells have been reported in the art. These are all useful in the methods according to the current invention. In certain embodiments of all aspects and embodiments, electroporation, nucleofection, or microinjection for nucleic acid transfer/transfection is used. In certain embodiments of all aspects and embodiments, an inorganic substance (such as, e.g., calcium phosphate/DNA co-precipitation), a cationic polymer (such as, e.g., polyethylenimine, DEAE-dextran), or a cationic lipid (lipofection) is used for nucleic acid transfer/transfection is used. Calcium phosphate and polyethylenimine are the most commonly used reagents for transfection for nucleic acid transfer in larger scales (see, e.g., Baldi et al., Biotechnol. Lett. 29 (2007) 677-684), whereof polyethylenimine is preferred.

The growth in serum-free suspension culture and improvement of efficiency and reproducibility of transfection conditions using PEI as a transfection reagent permits ready scale-up the AAV production using shake-flasks, wave, or stirred-tank bioreactors.

In certain embodiments of all aspects and embodiments, the nucleic acid (plasmid) is provided in a composition in combination with polyethylenimine (PEI), optionally in combination with cells. In certain embodiments, the composition includes a plasmid/PEI mixture, which has a plurality of components: (a) one or more plasmids comprising nucleic acids encoding AAV packaging proteins and/or nucleic acids encoding helper proteins; (b) a plasmid comprising a nucleic acid that encodes a protein or is transcribed into a transcript of interest; and (c) a polyethylenimine (PEI) solution. In certain embodiments, the plasmids are in a molar ratio range of about 1:0.01 to about 1:100, or are in a molar ratio range of about 100:1 to about 1:0.01, and the mixture of components (a), (b) and (c) has optionally been incubated for a period of time from about 10 seconds to about 4 hours.

In certain embodiments of all aspects and embodiments, the compositions further comprise cells. In certain embodiments, the cells are in contact with the plasmid/PEI mixture of components (a), (b) and/or (c).

In certain embodiments of all aspects and embodiments, the composition, optionally in combination with cells, further comprise free PEI. In certain embodiments, the cells are in contact with the free PEI.

In certain embodiments of all aspects and embodiments, the cells have been in contact with the mixture of components (a), (b) and/or (c) for at least about 4 hours, or about 4 hours to about 140 hours, or for about 4 hours to about 96 hours. In one preferred embodiment, the cells have been in contact with the mixture of components (a), (b) and/or (c) and optionally free PEI, for at least about 4 hours.

The composition may comprise further plasmids or/and cells. Such plasmids and cells may be in contact with free PEI. In certain embodiments, the plasmids and/or cells have been in contact with the free PEI for at least about 4 hours, or about 4 hours to about 140 hours, or for about 4 hours to about 96 hours.

The invention also provides methods for producing transfected cells. The method includes the steps of providing one or more plasmids; providing a solution comprising polyethylenimine (PEI); and mixing the plasmid(s) with the PEI solution to produce a plasmid/PEI mixture. In certain embodiments, such mixtures are incubated for a period in the range of about 10 seconds to about 4 hours. In such methods, cells are then contacted with the plasmid/PEI mixture to produce a plasmid/PEI cell culture; then free PEI is added to the plasmid/PEI cell culture produced to produce a free PEI/plasmid/PEI cell culture; and then the free PEI/plasmid/PEI cell culture produced is incubated for at least about 4 hours, thereby producing transfected cells. In certain embodiments, the plasmids comprise one or more or all of a rep open reading frame, a cap open reading frame, E1A, E1B, E2 and E4orf6 open reading frames and a nucleic acid that encodes a protein or is transcribed into a transcript of interest.

Further provided are methods for producing transfected cells that produce recombinant AAV vector or AAV particle, which include providing one or more plasmids comprising nucleic acids encoding AAV packaging proteins and/or nucleic acids encoding helper proteins; providing a plasmid comprising a nucleic acid that encodes a protein or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period in the range of about 10 seconds to about 4 hours); contacting mammalian cells with the plasmid/PEI mixture, to produce a plasmid/PEI cell culture; adding free PEI to the plasmid/PEI cell culture produced to produce a free PEI/plasmid/PEI cell culture; and incubating the free PEI/plasmid/PEI cell culture for at least about 4 hours, thereby producing transfected cells that produce recombinant AAV vector or particle comprising a nucleic acid that encodes a protein or is transcribed into a transcript of interest, whereby the mammalian cells have been obtained with a method according to the current invention.

Additionally provided are methods for producing recombinant AAV vector or AAV particle comprising a nucleic acid that encodes a protein or is transcribed into a transcript of interest, which includes providing one or more plasmids comprising nucleic acids encoding AAV packaging proteins and/or nucleic acids encoding helper proteins; providing a plasmid comprising a nucleic acid that encodes a protein of interest or is transcribed into a transcript of interest; providing a solution comprising polyethylenimine (PEI); mixing the aforementioned plasmids with the PEI solution, wherein the plasmids are in a molar ratio range of about 1:0.01 to about 1:100, or are in a molar ratio range of about 100:1 to about 1:0.01, to produce a plasmid/PEI mixture (and optionally incubating the plasmid/PEI mixture for a period of time in the range of about 10 seconds to about 4 hours); contacting mammalian cells with the plasmid/PEI mixture produced as described to produce a plasmid/PEI cell culture; adding free PEI to the plasmid/PEI cell culture produced as described to produce a free PEI/plasmid/PEI cell culture; incubating the plasmid/PEI cell culture or the free PEI/plasmid/PEI cell culture produced for at least about 4 hours to produce transfected cells; harvesting the transfected cells produced and/or culture medium from the transfected cells produced to produce a cell and/or culture medium harvest; lysing the cells and optionally isolating the recombinant AAV vector or particle from the cell and/or culture medium harvest lysate, whereby the mammalian cells have been obtained with a method according to the current invention; and thereby producing recombinant AAV vector or particle comprising a nucleic acid that encodes a protein or is transcribed into a transcript of interest.

Methods for producing recombinant AAV vectors or AAV particles using the method according to the current invention can include one or more further steps or features. An exemplary step or feature includes, but is not limited to, a step of harvesting the cultivated mammalian cells produced and/or harvesting the culture medium from the cultivated cells produced to produce a cell and/or culture medium harvest. An additional exemplary step or feature includes, but is not limited to lysing the harvested cells and optionally isolating the recombinant AAV vector or AAV particle from the cell and/or culture medium harvest lysate; whereby the mammalian cells prior to transfection have been obtained a method according to the current invention; and thereby producing recombinant AAV vector or AAV particle comprising a nucleic acid that encodes a protein or is transcribed into a transcript of interest.

In certain embodiments of all aspects and embodiments, PEI is added to the plasmids and/or cells at various time points. In certain embodiments, free PEI is added to the cells before, at the same time as, or after the plasmid/PEI mixture is contacted with the cells.

In certain embodiments of all aspects and embodiments, the cells are at particular densities and/or cell growth phases and/or viability when contacted with the plasmid/PEI mixture and/or when contacted with the free PEI. In one preferred embodiment, cells are at a density in the range of about 1*10\hat{ }5 cells/mL to about 1*10\hat{ }8 cells/mL when contacted with the plasmid/PEI mixture and/or when contacted with the free PEI. In certain embodiments, viability of the cells when contacted with the plasmid/PEI mixture or with the free PEI is about 60% or greater than 60%, or wherein the cells are in log phase growth when contacted with the plasmid/PEI mixture, or viability of the cells when contacted with the plasmid/PEI mixture or with the free PEI is about 90% or greater than 90%, or wherein the cells are in log phase growth when contacted with the plasmid/PEI mixture or with the free PEI.

In addition to PEI, valproic acid (VPA) can be used to improve transfection efficiency. VPA, a branched short-chain fatty acid and inhibits histone deacetylase activity. Due to this reason, it is commonly added to mammalian cell culture as an enhancer of recombinant protein production.

Encoded AAV packaging proteins include, in certain embodiments of all aspects and embodiments, AAV rep and/or AAV cap. Such AAV packaging proteins include, in certain embodiments of all aspects and embodiments, AAV rep and/or AAV cap proteins of any AAV serotype.

Encoded helper proteins include, in certain embodiments of all aspects and embodiments, adenovirus E1A and E1B, adenovirus E2 and/or E4, VA RNA, and/or non-AAV helper proteins.

In certain embodiments of all aspects and embodiments, the nucleic acids (plasmids) are used at particular amounts or ratios. In certain embodiments, the total amount of plasmid comprising the nucleic acid that encodes a protein or is transcribed into a transcript of interest and the one or more plasmids comprising nucleic acids encoding AAV packaging proteins and/or nucleic acids encoding helper proteins is in the range of about 0.1 g to about 15 g per mL of cells. In certain embodiments, the molar ratio of the plasmid comprising the nucleic acid that encodes a protein or is transcribed into a transcript of interest to the one or more plasmids comprising nucleic acids encoding AAV packaging proteins and/or nucleic acids encoding helper proteins is in the range of about 1:5 to about 1:1, or is in the range of about 1:1 to about 5:1.

In certain embodiments of all aspects and embodiments, a first plasmid comprises the nucleic acids encoding AAV packaging proteins and a second plasmid comprises the nucleic acids encoding helper proteins.

In certain embodiments of all aspects and embodiments, the molar ratio of the plasmid comprising the nucleic acid that encodes a protein or is transcribed into a transcript of interest to a first plasmid comprising the nucleic acids encoding AAV packaging proteins to a second plasmid comprising the nucleic acids encoding helper proteins is in the range of about 1-5:1:1, or 1:1-5:1, or 1:1:1-5 in co-transfection.

In certain embodiments of all aspects and embodiments, the cell is a eukaryotic cell. In certain embodiments, the eukaryotic cell is a mammalian cell. In one preferred embodiment, the cell is a HEK293 cell or a CHO cell.

The cultivation can be performed using the generally used conditions for the cultivation of eukaryotic cells of about 37° C., 95% humidity and 8 vol.-% CO₂. The cultivation can be performed in serum containing or serum free medium, in adherent culture or in suspension culture. The suspension cultivation can be performed in any fermentation vessel, such as, e.g., in stirred tank reactors, wave reactors, rocking bioreactors, shaker vessels or spinner vessels or so called roller bottles. Transfection can be performed in high throughput format and screening, respectively, e.g. in a 96 or 384 well format.

Methods according to the current invention include AAV particles of any serotype, or a variant thereof. In certain embodiments of all aspects and embodiments, a recombinant AAV particle comprises any of AAV serotypes 1-12, an AAV VP1, VP2 and/or VP3 capsid protein, or a modified or variant AAV VP1, VP2 and/or VP3 capsid protein, or wild-type AAV VP1, VP2 and/or VP3 capsid protein. In certain embodiments of all aspects and embodiments, an AAV particle comprises an AAV serotype or an AAV pseudotype, where the AAV pseudotype comprises an AAV capsid serotype different from an ITR serotype.

Methods according to the invention that provide or include AAV vectors or particles can also include other elements. Examples of such elements include but are not limited to: an intron, an expression control element, one or more adeno-associated virus (AAV) inverted terminal repeats (ITRs) and/or a filler/stuffer polynucleotide sequence. Such elements can be within or flank the nucleic acid that encodes a protein or is transcribed into a transcript of interest, or the expression control element can be operably linked to nucleic acid that encodes a protein or is transcribed into a transcript of interest, or the AAV ITR(s) can flank the 5′- or 3′-terminus of nucleic acid that encodes a protein or is transcribed into a transcript of interest, or the filler polynucleotide sequence can flank the 5′- or 3′-terminus of nucleic acid that encodes a protein or is transcribed into a transcript of interest.

Expression control elements include constitutive or regulatable control elements, such as a tissue-specific expression control element or promoter.

ITRs can be any of AAV2 or AAV6 or AAV8 or AAV9 serotypes, or a combination thereof. AAV particles can include any VP1, VP2 and/or VP3 capsid protein having 75% or more sequence identity to any of AAV1, AAV2, AAV3, AAV4, AAV5, AAV6, AAV10, AAV11, AAV-2i8, AAV rh74 or AAV 7m8 VP1, VP2 and/or VP3 capsid proteins, or comprises a modified or variant VP1, VP2 and/or VP3 capsid protein selected from any of: AAV1, AAV2, AAV3, AAV4, AAV5, AAV6, AAV10, AAV11, AAV-2i8, AAV rh74 and AAV 7m8 AAV serotypes.

Following production of recombinant viral (e.g., AAV) particles as set forth herein, if desired, the viral (e.g., rAAV) particles can be purified and/or isolated from host cells using a variety of conventional methods. Such methods include column chromatography, CsCl gradients, iodixanol gradient and the like.

For example, a plurality of column purification steps such as purification over an anion exchange column, an affinity column and/or a cation exchange column can be used. (See, e.g., WO 02/12455 and US 2003/0207439). Alternatively, or in addition, an iodixanol or CsCl gradient steps can be used (see, e.g., US 2012/0135515; and US 2013/0072548). Further, if the use of infectious virus is employed to express the packaging and/or helper proteins, residual virus can be inactivated, using various methods. For example, adenovirus can be inactivated by heating to temperatures of approximately 60° C. for, e.g., 20 minutes or more. This treatment effectively inactivates the helper virus since AAV is heat stable while the helper adenovirus is heat labile.

An objective in the rAAV vector production and purification systems is to implement strategies to minimize/control the generation of production related impurities such as proteins, nucleic acids, and vector-related impurities, including wild-type/pseudo wild-type AAV species (wtAAV) and AAV-encapsulated residual DNA impurities.

Considering that the rAAV particle represents only a minor fraction of the biomass, rAAV particles need to be purified to a level of purity, which can be used as a clinical human gene therapy product (see, e.g., Smith P. H., et al., Mo. Therapy 7 (2003) 8348; Chadeuf G., et al, Mo. Therapy 12 (2005) 744; report from the CHMP gene therapy expert group meeting, European Medicines Agency EMEA/CHMP 2005, 183989/2004).

As an initial step, typically the cultivated cells that produce the rAAV particles are harvested, optionally in combination with harvesting cell culture supernatant (medium) in which the cells (suspension or adherent) producing rAAV particles have been cultured. The harvested cells and optionally cell culture supernatant may be used as is, as appropriate, lysed or concentrated. Further, if infection is employed to express helper functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

Cells and/or supernatant of the harvest are lysed by disrupting the cells, for example, by chemical or physical means, such as detergent, microfluidization and/or homogenization, to release the rAAV particles. Concurrently during cell lysis or subsequently after cell lysis, a nuclease, such as, e.g., benzonase, is added to degrade contaminating DNA. Typically, the resulting lysate is clarified to remove cell debris, e.g. by filtering or centrifuging, to render a clarified cell lysate. In a particular example, lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (comprising rAAV vectors as well as empty capsids) and production/process related impurities, such as soluble cellular components from the host cells that can include, inter alia, cellular proteins, lipids, and/or nucleic acids, and cell culture medium components. The optionally clarified lysate is then subjected to purification steps to purify AAV particles (comprising rAAV vectors) from impurities using chromatography. The clarified lysate may be diluted or concentrated with an appropriate buffer prior to the first chromatography step.

After cell lysis, optional clarifying, and optional dilution or concentration, a plurality of subsequent and sequential chromatography steps can be used to purify rAAV particles.

The removal of empty capsids from full ones, for example, during downstream processing is based on their different isoelectric points (pI) in anion exchange chromatography. The average calculated pI across all serotypes is 5.9 for full capsids and 6.3 for empty capsids (Venkatakrishnan, B., et al., J. Virol. 87 (2013) 4974-4984).

A first chromatography step may be cation exchange chromatography or anion exchange chromatography. If the first chromatography step is cation exchange chromatography the second chromatography step can be anion exchange chromatography or size exclusion chromatography (SEC). Thus, in certain embodiments of all aspects and embodiments, rAAV particle purification is via cation exchange chromatography, followed by purification via anion exchange chromatography.

Alternatively, if the first chromatography step is cation exchange chromatography the second chromatography step can be size exclusion chromatography (SEC). Thus, in certain embodiments of all aspects and embodiments, rAAV particle purification is via cation exchange chromatography, followed by purification via size exclusion chromatography (SEC).

Still alternatively, a first chromatography step may be affinity chromatography. If the first chromatography step is affinity chromatography the second chromatography step can be anion exchange chromatography. Thus, in certain embodiments of all aspects and embodiments, rAAV particle purification is via affinity chromatography, followed by purification via anion exchange chromatography.

Optionally, a third chromatography can be added to the foregoing chromatography steps. Typically, the optional third chromatography step follows cation exchange, anion exchange, size exclusion or affinity chromatography.

Thus, in certain embodiments of all aspects and embodiments, rAAV particle purification is via cation exchange chromatography, followed by purification via anion exchange chromatography, followed by purification via size exclusion chromatography (SEC).

In addition, in certain embodiments of all aspects and embodiments, further rAAV particle purification is via cation exchange chromatography, followed by purification via size exclusion chromatography (SEC), followed by purification via anion exchange chromatography.

In yet further embodiments of all aspects and embodiments, rAAV particle purification is via affinity chromatography, followed by purification via anion exchange chromatography, followed by purification via size exclusion chromatography (SEC).

In yet further embodiments of all aspects and embodiments, rAAV particle purification is via affinity chromatography, followed by purification via size exclusion chromatography (SEC), followed by purification via anion exchange chromatography.

Cation exchange chromatography functions to separate the AAV particles from cellular and other components present in the clarified lysate and/or column eluate from an affinity or size exclusion chromatography. Examples of strong cation exchange resins capable of binding rAAV particles over a wide pH range include, without limitation, any sulfonic acid based resin as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, MA, USA). Additional examples include Capto S, Capto S ImpAct, Capto S ImpRes (strong cation exchangers available from GE Healthcare, Marlborough, MA, USA), and commercial DOWEX®, AMBERLITE®, and AMBERLYST® families of resins available from Aldrich Chemical Company (Milliwaukee, WI, USA). Weak cation exchange resins include, without limitation, any carboxylic acid based resin. Exemplary cation exchange resins include carboxymethyl (CM), phospho (based on the phosphate functional group), methyl sulfonate (S) and sulfopropyl (SP) resins.

Anion exchange chromatography functions to separate AAV particles from proteins, cellular and other components present in the clarified lysate and/or column eluate from an affinity or cation exchange or size exclusion chromatography. Anion exchange chromatography can also be used to reduce and thereby control the amount of empty capsids in the eluate. For example, the anion exchange column having rAAV particle bound thereto can be washed with a solution comprising NaCl at a modest concentration (e.g., about 100-125 mM, such as 110-115 mM) and a portion of the empty capsids can be eluted in the flow through without substantial elution of the rAAV particles. Subsequently, rAAV particles bound to the anion exchange column can be eluted using a solution comprising NaCl at a higher concentration (e.g., about 130-300 mM NaCl), thereby producing a column eluate with reduced or depleted amounts of empty capsids and proportionally increased amounts of rAAV particles comprising an rAAV vector.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography materials include, without limitation, MACRO PREP Q (strong anion-exchanger available from BioRad, Hercules, CA, USA); UNOSPHERE Q (strong anion-exchanger available from BioRad, Hercules, CA, USA); POROS 50HQ (strong anion-exchanger available from Applied Biosystems, Foster City, CA, USA); POROS XQ (strong anion-exchanger available from Applied Biosystems, Foster City, CA, USA); POROS SOD (weak anion-exchanger available from Applied Biosystems, Foster City, CA, USA); POROS 50PI (weak anion-exchanger available from Applied Biosystems, Foster City, CA, USA); Capto Q, Capto XQ, Capto Q ImpRes, and SOURCE 30Q (strong anion-exchanger available from GE healthcare, Marlborough, MA, USA); DEAE SEPHAROSE (weak anion-exchanger available from Amersham Biosciences, Piscataway, NJ, USA); Q SEPHAROSE (strong anion-exchanger available from Amersham Biosciences, Piscataway, NJ, USA). Additional exemplary anion exchange resins include aminoethyl (AE), diethylaminoethyl (DEAE), diethylaminopropyl (DEPE) and quaternary amino ethyl (QAE).

A manufacturing process to purify recombinant AAV particles intended as a product to treat human disease should achieve the following objectives: 1) consistent particle purity, potency and safety; 2) manufacturing process scalability; and 3) acceptable cost of manufacturing.

Exemplary processes for recombinant AAV particle purification are reported in WO 2019/006390.

The below outlined recombinant adeno-associated virus particle (rAAV particle) purification and production methods are scalable up to large scale. For example, to a suspension culture of 5, 10, 10-20, 20-50, 50-100, 100-200 or more liters volume. The recombinant adeno-associated virus particle purification and production methods are applicable to a wide variety of AAV serotypes/capsid variants.

In certain embodiments of all aspects and embodiments, the purification of rAAV particles comprises the steps of:
a) harvesting mammalian cells and/or cell culture supernatant comprising rAAV particles to produce a harvest;
b) optionally concentrating the harvest produced in step (a) to produce a concentrated harvest;
c) lysing the harvest produced in step (a) or the concentrated harvest produced in step (b) to produce a lysate;
d) treating the lysate produced in step (c) to reduce contaminating nucleic acid in the lysate thereby producing a nucleic acid reduced lysate;
e) optionally filtering the nucleic acid reduced lysate produced in step (d) to produce a clarified lysate, and optionally diluting the clarified lysate to produce a diluted clarified lysate;
f) subjecting the nucleic acid reduced lysate of step (d), the clarified lysate of step (e), or the diluted clarified lysate produced in step (e) to a cation exchange column chromatography to produce a column eluate comprising rAAV particles, thereby separating rAAV particles from protein impurities or other production/process related impurities, and optionally diluting the column eluate to produce a diluted column eluate;
g) subjecting the column eluate or the diluted column eluate produced in step (f) to an anion exchange chromatography to produce a second column eluate comprising rAAV particles, thereby separating rAAV particles from protein impurities or production/process related impurities, and optionally concentrating the second column eluate to produce a concentrated second column eluate;
h) subjecting the second column eluate or the concentrated second column eluate produced in step (g) to a size exclusion column chromatography (SEC) to produce a third column eluate comprising rAAV particles, thereby separating rAAV particles from protein impurities or production/process related impurities, and optionally concentrating the third column eluate to produce a concentrated third column eluate; and
i) filtering the third column eluate or the concentrated third column eluate produced in step (h), thereby producing purified rAAV particles;
whereby the mammalian cells prior to transfection/production have been obtained with the method according to the invention.

In certain embodiments, steps (a) to (f) are maintained and combined with the following steps:
g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a concentrated second column eluate;
h) subjecting the second column eluate or the diluted second column eluate produced in step (g) to an anion exchange chromatography to produce a third column eluate comprising rAAV particles thereby separating rAAV particles from protein impurities production/process related impurities and optionally diluting the third column eluate to produce a diluted third column eluate; and
i) filtering the third column eluate or the concentrated third column eluate produced in step (h), thereby producing purified rAAV particles.

In certain embodiments, steps (a) to (g) are maintained and combined with the following step:
h) filtering the second column eluate or the concentrated second column eluate produced in step (g), thereby producing purified rAAV particles.

In embodiment, steps (a) to (e) are maintained and combined with the following steps:
f) subjecting the nucleic acid reduced lysate in step (d), or clarified lysate or diluted clarified lysate produced in step (e) to an AAV affinity column chromatography to produce a column eluate comprising rAAV particles, thereby separating rAAV particles from protein impurities or other production/process related impurities, and optionally concentrating the column eluate to produce a concentrated column eluate;
g) subjecting the column eluate or the concentrated column eluate produced in step (f) to a size exclusion column chromatography (SEC) to produce a second column eluate comprising rAAV particles, thereby separating rAAV particles from protein impurities or other production/process related impurities, and optionally diluting the second column eluate to produce a diluted second column eluate;
h) optionally subjecting the second column eluate or the diluted second column eluate produced in step (g) to an anion exchange chromatography to produce a third column eluate comprising rAAV particles, thereby separating rAAV particles from protein impurities or other production/process related impurities, and optionally diluting the third column eluate to produce a diluted third column eluate; and
i) filtering the second column eluate or the diluted second column eluate produced in step (g), or filtering the third column eluate or the concentrated third column eluate produced in step (h), thereby producing purified rAAV particles.

In certain embodiments of all aspects and embodiments, concentrating of step (b) and/or step (f) and/or step (g) and/or step (h) is via ultrafiltration/diafiltration, such as by tangential flow filtration (TFF).

In certain embodiments of all aspects and embodiments, concentrating of step (b) reduces the volume of the harvested cells and cell culture supernatant by about 2-20 fold.

In certain embodiments of all aspects and embodiments, concentrating of step (f) and/or step (g) and/or step (h) reduces the volume of the column eluate by about 5-20 fold.

In certain embodiments of all aspects and embodiments, lysing of the harvest produced in step (a) or the concentrated harvest produced in step (b) is by physical or chemical means. Non-limiting examples of physical means include microfluidization and homogenization. Non-limiting examples of chemical means include detergents. Detergents include non-ionic and ionic detergents. Non-limiting examples of non-ionic detergents include Triton X-100. Non-limiting examples of detergent concentration is between about 0.1 and 1.0% (v/v) or (w/v), inclusive.

In certain embodiments of all aspects and embodiments, step (d) comprises treating with a nuclease thereby reducing contaminating nucleic acid. Non-limiting examples of a nuclease include benzonase.

In certain embodiments of all aspects and embodiments, filtering of the clarified lysate or the diluted clarified lysate of step (e) is via a filter. Non-limiting examples of filters are those having a pore diameter of between about 0.1 and 10.0 microns, inclusive.

In certain embodiments of all aspects and embodiments, diluting of the clarified lysate of step (e) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, diluting of the column eluate of step (f) or the second column eluate of step (g) is with an aqueous buffered phosphate, acetate or Tris solution. Non-limiting examples of solution pH are between about pH 4.0 and pH 7.4, inclusive. Non-limiting examples of Tris solution pH are greater than pH 7.5, such as between about pH 8.0 and pH 9.0, inclusive.

In certain embodiments of all aspects and embodiments, the rAAV particles resulting from step (i) are formulated with a surfactant to produce a rAAV particle formulation.

In certain embodiments of all aspects and embodiments, the anion exchange column chromatography of step (f), (g) and/or (h) comprises polyethylene glycol (PEG) modulated column chromatography.

In certain embodiments of all aspects and embodiments, the anion exchange column chromatography of step (g) and/or (h) is washed with a PEG solution prior to elution of the rAAV particles from the column.

In certain embodiments of all aspects and embodiments, the PEG has an average molecular weight in a range of about 1,000 g/mol to 80,000 g/mol, inclusive.

In certain embodiments of all aspects and embodiments, the PEG is at a concentration of about 4% to about 10% (w/v), inclusive.

In certain embodiments of all aspects and embodiments, the anion exchange column of step (g) and/or (h) is washed with an aqueous surfactant solution prior to elution of the rAAV particles from the column.

In certain embodiments of all aspects and embodiments, the cation exchange column of step (f) is washed with a surfactant solution prior to elution of the rAAV particles from the column.

In certain embodiments of all aspects and embodiments, the PEG solution and/or the surfactant solution comprises an aqueous Tris-HCl/NaCl buffer, an aqueous phosphate/NaCl buffer, or an aqueous acetate/NaCl buffer.

In certain embodiments of all aspects and embodiments, NaCl concentration in the buffer or solution is in a range of between about 20-300 mM NaCl, inclusive, or between about 50-250 mM NaCl, inclusive.

In certain embodiments of all aspects and embodiments, the surfactant comprises a cationic or anionic surfactant.

In certain embodiments of all aspects and embodiments, the surfactant comprises a twelve carbon chained surfactant.

In certain embodiments of all aspects and embodiments, the surfactant comprises Dodecyltrimethylammonium chloride (DTAC) or Sarkosyl.

In certain embodiments of all aspects and embodiments, the rAAV particles are eluted from the anion exchange column of step (f), (g) and/or (h) with an aqueous Tris-HCl/NaCl buffer.

In certain embodiments of all aspects and embodiments, the Tris-HCl/NaCl buffer comprises 100-400 mM NaCl, inclusive, optionally at a pH in a range of about pH 7.5 to about pH 9.0, inclusive.

In certain embodiments of all aspects and embodiments, the anion exchange column of step (f), (g) and/or (h) is washed with an aqueous Tris-HCl/NaCl buffer.

In certain embodiments of all aspects and embodiments, the NaCl concentration in the aqueous Tris-HCl/NaCl buffer is in a range of about 75-125 mM, inclusive.

In certain embodiments of all aspects and embodiments, the aqueous Tris-HCl/NaCl buffer has a pH from about pH 7.5 to about pH 9.0, inclusive.

In certain embodiments of all aspects and embodiments, the anion exchange column of step (f), (g) and/or (h) is washed one or more times to reduce the amount of empty capsids in the second or third column eluate.

In certain embodiments of all aspects and embodiments, the anion exchange column wash removes empty capsids from the column prior to rAAV particle elution and/or instead of rAAV particle elution, thereby reducing the amount of empty capsids in the second or third column eluate.

In certain embodiments of all aspects and embodiments, the anion exchange column wash removes at least about 50% of the total empty capsids from the column prior to rAAV particle elution and/or instead of rAAV particle elution, thereby reducing the amount of empty capsids in the second or third column eluate by about 50%.

In certain embodiments of all aspects and embodiments, the NaCl concentration in the aqueous Tris-HCl/NaCl buffer is in a range of about 110-120 mM, inclusive.

In certain embodiments of all aspects and embodiments, ratios and/or amounts of the rAAV particles and empty capsids eluted are controlled by a wash buffer.

In certain embodiments of all aspects and embodiments, the rAAV particles are eluted from the cation exchange column of step (f) in an aqueous phosphate/NaCl buffer, or an aqueous acetate/NaCl buffer. Non-limiting NaCl concentration in a buffer is in a range of about 125-500 mM NaCl, inclusive. Non-limiting examples of buffer pH are between about pH 5.5 to about pH 7.5, inclusive.

In certain embodiments of all aspects and embodiments, the anion exchange column of step (f), (g) and/or (h) comprises a quaternary ammonium functional group such as quaternized polyethylenimine.

In certain embodiments of all aspects and embodiments, the size exclusion column (SEC) of step (g) and/or (h) has a separation/fractionation range (molecular weight) from about 10,000 g/mol to about 600,000 g/mol, inclusive.

In certain embodiments of all aspects and embodiments, the cation exchange column of step (f) comprises a sulfonic acid or functional group such as sulphopropyl.

In certain embodiments of all aspects and embodiments, the AAV affinity column comprises a protein or ligand that binds to AAV capsid protein. Non-limiting examples of a protein include an antibody that binds to AAV capsid protein. More specific non-limiting examples include a single-chain Llama antibody (Camelid) that binds to AAV capsid protein.

In certain embodiments of all aspects and embodiments, the method excludes a step of cesium chloride gradient ultracentrifugation.

In certain embodiments of all aspects and embodiments, the method recovers approximately 50-90% of the total rAAV particles from the harvest produced in step (a) or the concentrated harvest produced in step (b).

In certain embodiments of all aspects and embodiments, the method produces rAAV particles having a greater purity than rAAV particles produced or purified by a single AAV affinity column purification.

In certain embodiments of all aspects and embodiments, steps (c) and (d) are performed substantially concurrently.

In certain embodiments of all aspects and embodiments, the NaCl concentration is adjusted to be in a range of about 100-400 mM NaCl, inclusive, or in a range of about 140-300 mM NaCl, inclusive, after step (c) but prior to step (f).

In certain embodiments of all aspects and embodiments, the cells are suspension growing or adherent growing cells.

In certain embodiments of all aspects and embodiments, the cells are mammalian cells. Non-limiting examples include HEK cells, such as HEK-293 cells, and CHO cells, such as CHO-K1 cells.

Methods to determine infectious titer of rAAV particles containing a transgene are known in the art (see, e.g., Zhen et al., Hum. Gene Ther. 15 (2004) 709). Methods for assaying for empty capsids and rAAV particles with packaged transgenes are known (see, e.g., Grimm et al., Gene Therapy 6 (1999) 1322-1330; Sommer et al., Malec. Ther. 7 (2003) 122-128).

To determine the presence or amount of degraded/denatured capsid, purified rAAV particle can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (see, e.g., Wobus et al., J. Viral. 74 (2000) 9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

### Perfusion

Due to a continuous exchange of the medium in the perfusion bioreactor, both the continuous supply of nutrients via the feed and the continuous removal of waste products via the permeate are provided. Thus, the accumulation of waste products negatively influencing cell growth and viability, which are observed in fed-batch processes, can be overcome. Furthermore, the continuous medium renewal provides a more constant cell environment, which is beneficial for cell growth, health, metabolism, and product quality (Chotteau, V., “Perfusion Processes”, in Animal Cell Culture, M. Al-Rubeai, (Ed.) Cham: Springer International Publishing, 2015, pp. 407-443; Karst, D. J., et al., Curr. Opin. Biotechnol. 53 (2018) 76-84; Walther, J., et al., Biotechnol. J., 14 (2019) e1700733).

In the context of viral vector production, an increasing number of studies are focusing on perfusion technologies to further increase the production yield, improve the full-to-empty (F/E) ratio, and reduce the cost of viral vector production. Perfusion mode has already been validated as an advantageous strategy for adenoviral, retroviral, lentiviral, and AAV vector production in HEK293 cells (Ghani, K., et al., Biotechnol. Bioeng. 95 (2006) 653-660; Henry, O., et al., J. Process Cont. 17 (2007) 241-251; Henry, O., et al., Biotechnol. Bioeng. 86 (2004) 765-774; Ansorge, S., et al., J. Gene Med. 11 (2009) 868-876; Merten, O. W., J. Gene Med. 6 (2004) S105-S124; Benskey, M. J., et al., Hum. Gene Ther. Meth. 27 (2016) 32-45; Coronel, J., et al., Gen. Engin. Biotechnol. News 41 (2021) S23-S23).

For perfusion in viral vector production and in contrast to classical biotechnological production of recombinant proteins, the most important goal is no longer to achieve the highest possible cell density and to maintain long production phases to increase volumetric productivity. Instead, cells need to be maintained around 2×10\hat{ }6 cells/mL for efficient transfection. Higher cell densities are more difficult to transfect due to the widely described cell density effect (CDE) (Petiot, E., et al., Vaccine 33 (2015) 5974-5981; Le Ru, A., et al., Vaccine 28 (2010) 3661-3671; Bernal, V., et al., Biotechnol. Bioeng. 104 (2009) 162-180). Transfection efficiency and productivity decrease when performed at higher cell densities (Lavado-Garcia, J., et al., Front. Bioeng. Biotechnol. 8 (2020) Article 617).

Continuous perfusion is used to constantly renew medium while a high proportion of cells is kept in an exponential growth phase for efficient transfection (Henry, O., et al., Biotechnol. Bioeng. 86 (2004) 765-774; Cortin, V., et al., Biotechnol. Prog. 20 (2004) 858-863). Accordingly, viral vectors are produced up to a certain limited process span, and released viral vectors into the medium can be rapidly harvested.

The perfusion rate describes the rate of medium renewal in a perfusion process and is commonly expressed as the volume of added medium per bioreactor working volume per day (vvd). In literature, different strategies for perfusion rate control have been described, including strategies based on the cell density, the concentration of the main substrate or a by-product.

When the medium renewal in a perfusion process is normalized to the cell density, it is expressed as cell-specific perfusion rate. Perfusion control based on CSPR, as described by Dowd et al., directly links the perfusion rate to the cell density (Cytotechnol. 42 (2003) 35-45). A lower CSPR means more cells can be sustained with a certain amount of medium. Hence, a consistent microenvironment to the cells in culture can be ensured by keeping the CSPR constant during the process.

To enhance the economic potential of a process, the volumetric perfusion rate should be minimized. However, it must be considered that if the CSPR falls below a lower limit, poor cell growth, a decrease in viability, product degradation, or a decrease in specific productivity might compromise production or make it impossible. Thus, optimal CSPR close to the minimal CSPR has to be adjusted during process development (Konstantinov, K., et al., Adv. Biochem. Eng. Biotechnol. 101 (2006) 75-98; Bielser, J. M., et al., Biotechnol. Prog. 36 (2020) e3026).

### Inoculation Train

A conventional inoculation train usually starts by thawing a small amount of frozen cells, such as, e.g., a 1 mL cryopreserved vial from the working cell bank. Subsequently, the cell culture is expanded into larger cultivation systems. For this purpose, cells are cultivated in shake flasks, spinner flasks, and stirred-tank bioreactors with increasing volume. The production bioreactor is often referred to as N-stage whereas the bioreactors before the production stage are named N-1, N-2, N-3 in decreasing order.

High-density cell banking and the use of disposable systems improve operational success by reducing the duration of the inoculation train and the contamination potential. This allows simplification and reduction of the inoculation train, resulting in reduced process complexity, operating times, and an increased yield.

### Dielectric Spectroscopy

Dielectric spectroscopy, which is also referred to as capacitance measurement or radio-frequency impedance, is a method primarily used for online monitoring of viable cell density or viable cell volume. The measurement principle of this tool is based on the characteristic of viable cells to have an intact non-conducting plasma membrane. This allows them to store electrical charge, which makes viable cells in aqueous ionic suspension behave like small capacitors when introduced into a periodically alternating electrical field in the radio frequency range (0.1-10 MHz).

The movement of intracellular and extracellular ions is constrained by the plasma membranes, which act as an insulating physical barrier. This results in polarization, which is the charge separation across the membrane at the poles of the cell. The magnitude of this polarization is measured by the capacitance ΔC in pico-Farads (pF).

Thus by measuring the capacitance of the suspension, its viable cell density can be estimated (J. P. Carvell and J. E. Dowd, Cytotechnol. 50 (2006) 35-48; Justice, C., et al., Biotechnol. Adv. 29 (2011) 391-401; Cole, H. E., et al., Processes 3 (2015) 384-405).

For most cells, this sigmoidal curve is centered between 0.5 and 3 MHz around the critical frequency ƒc. This is the frequency, at which the fall in capacitance is half completed (Cannizzaro, C., et al., Biotechnol. Bioeng. 84 (2003) 597-610). Usually, dielectric spectroscopy is performed close to this frequency because here the capacitance is independent of cell size (Justice, C., supra).

Capacity increases with increasing volume fraction of the cells, as there are more polarized membranes, which, in turn, give a higher measured capacitance. Generally, capacitance is not only dependent on the VCD, but also the cell type, the cell size, and the physiological state of the cell.

For VCD prediction, a mathematical model is necessary to correlate the capacitance signal with offline biomass data—the capacitance ΔC is multiplied by the cell constant K, which is given in 1/cm to obtain the absolute permittivity ε in pF/cm:

$$ε = Δ C * K \qquad ( 1 )$$

The value of biocapacitance for the control of perfusion processes, where perfusion feeding was automatically adjusted based on the live cell concentration from the online capacitance probe, has been demonstrated in several studies (Carvell and Dowd, supra; Cannizzaro, C., et al. supra). In addition, dielectric spectroscopy is emerging as a relevant tool for monitoring viral vector production processes, since it provides critical information on the physiological state of the cells (Pais, D. A. M., et al., Processes 8 (2020) 1456; Escandell, J. M., et al., Curr. Opin. Biotechnol. 74 (2022) 271-277).

### Embodiments of the Method According to the Invention

A scale up of the production process for recombinant AAV particles to 100 L, 500 L or even 1,000 L presents significant challenges, both for cell expansion as well as plasmid transfection.

Achieving a high capsid titer is the aim in recombinant AAV particle production. Thus, producing a large amount of cell mass resulting in the production of empty capsids is not suitable to fulfill this aim.

Therefore, the focus in perfusion processes for the production of viral vectors is not on achieving high cell densities, but on alleviating nutrient limitations by ensuring constant medium replacement.

A variety of cell retention devices is available for cell retention in processes for manufacturing viral vectors. The most commonly used are Alternating Tangential Flow Filtration (ATF), Tangential Flow Filtration (TFF), acoustic filters, and membrane-based internal retention systems.

ATF technology uses a hollow fiber module connected to the bioreactor via a single port. With a diaphragm pump, the cell suspension is pumped through the hollow fiber module where cells are retained and a clear supernatant containing the product of interest is harvested. Subsequently, to reduce membrane fouling, the flow is reversed, and the remaining cell suspension is flushed back into the bioreactor. The diaphragm pump generates a rapid low-shear flow, ensuring that cells quickly return to the bioreactor. Recently, Coronel et al. have shown that perfusion with an ATF system can be applied to increase rAAV particle production (Gen. Engin. Biotechnol. News 41 (2021) S23-S23).

However, in one specific embodiment of the method according to the invention, perfusion is not done by ATF.

In more detail, TFF technology is using a comparable hollow fiber system as ATF, but a low shear circulation pump instead of the diaphragm pump and two bioreactor ports. One to draw liquid from the bioreactor and the other to return liquid from the external cell retention device to the bioreactor. Different TFF systems are commercially available (Xcellerex APS™ by Cytiva, KML100™ by Repligen or Cellicon™ by Merck) for this application.

Suspension-based cell cultures were an important step from adherent cultures to provide scalability. However, cell densities per volume are generally lower compared to adherent cells. Therefore, high cell densities per volume have to be reached to approximate transfection efficiency in adherent cultures. Furthermore, consistently good cell growth and viabilities are required throughout the whole inoculation train and production process to ensure high productivity. With increasing production volume this becomes more difficult, for example, with respect to sufficient gas input, efficient mixing, and supply of nutrients. Therefore, the cells need to adapt to a changing environment. Moreover, every change in cultivation parameters triggers the cells' internal control mechanisms and activates responses, which possibly results in lower cell and product yields or increased by-product formation.

In addition, regarding the transfection process, often a decrease of transfection efficiency during scale-up is experienced.

In order to improve the production of recombinant AAV particles, there is a necessity to improve the inoculation train in order to prevent substrate depletion and high accumulation of waste products.

This has been achieved by ensuring the optimal physiological state of the cells before transfection. Thereby high transfection efficiencies were enabled. This has been achieved by reducing the proportion of spent medium in the production bioreactor from about 40% to about 20%. Consequently, lower concentrations of inhibitory waste products were present and at the same time the concentration of nutrients was increased. This allowed for an improvement of the cellular growth rate at the beginning of the N-process from about 0.3/d to about 1.0/d. In addition, the inoculation train could be simplified by omitting one bioreactor. Thereby the process complexity was reduced. Furthermore, by using automated perfusion based on online viable cell density (VCD) measurement, e.g. with dielectric spectroscopy, and the application of a cell-specific perfusion rate (CSPR), the required perfusion medium could be reduced by about one third.

In addition, by using the method according to the current invention the cell density at transfection in the main fermenter, i.e. the production bioreactor, could be controlled more precisely. Thereby the so-called cell density effect, i.e. the exceedance of the cell density at transfection, which results in lower production yields in AAV production, could be reduced or even prevented.

The invention is based, at least in part, on the finding that for the production of AAV particles it is advantageous that the host cells have been propagated prior to transfection and, thus, production of the AAV particle using perfusion. In more detail, it has been found that it is advantageous for recombinant production of AAV particles that the mammalian cell is propagated in the pre-culture using perfusion. The advantage of the current invention being that mammalian cells propagated using perfusion prior to transfection maintain grow with higher growth rate with higher mitotic activity and show a higher viability after transfection, i.e. during the production cultivation. Thereby, while having the same inoculation cell density a higher titer can be achieved compared to a cultivation wherein mammalian cells are used that have been cultivated prior to transfection exclusively using batch- or fed-batch methods.

The invention is based, at least in part, on the finding that for producing AAV particles in a cell culture starting from low cell densities it is advantageous to propagate the mammalian cell by perfusion and dilute the propagated mammalian cell prior to transfection with the transgene encoding nucleic acid.

It has been found that without using perfusion nutrients are excessively reduced and waste products accumulate to such amounts during pre-culture that the obtained cells are severely impacted and no longer suitable for subsequent AAV particle production due to impaired cell growth in the cultivation.

In the following examples, parameters, such as cell line, AAV serotype, medium, transfection reagent, as well as process parameters, such as temperature, pH, and pO2, were kept constant or highly similar allowing for a direct comparison of the results obtained with the different processes. Thus, the observed improvement is directly resulting from the difference of the cultivation conditions, i.e. the feeding, used for the propagation of the cells prior to transfection.

In a comparative example, recombinant AAV particle production was done at a 100 L scale.

The main fermenter is set to be inoculated with a cell density of 20*10\hat{ }5 cells/mL and a starting volume of 60 L, a target of 1.2*10\hat{ }11 cells had to be reached after the N-1 fermentation. With a working volume of 25 L in N-1, a final density of at least 48*10\hat{ }5 cells/mL was required in N-1. Since the cells were known to double almost every day and to add a safety margin, inoculation of N-1 was performed with 8*10\hat{ }5 cells/mL to reach a final density of approximately 60*10\hat{ }5 cells/mL. The N-2 fermentation was inoculated with 5*10\hat{ }5 cells/mL according to the same considerations to reach about 40*10\hat{ }5 cells/mL after three days. The pH in the inoculation train was chosen to support high cell growth while limiting glucose consumption and accumulation of toxic waste products. In the main fermenter, the pH was slightly increased to increase AAV production as well as their release into the culture medium.

However, significant problems arose in the N-1 25 L Rocker as the cell growth and viability deteriorated subsequently to the N-2 stage. Moreover, the cells did not recover after they were transferred to the main fermenter as they were already too damaged. This meant that no transfection could be performed since very high viability is required for efficient transfection.

In the N-1 stage a poorer cell growth than in the N-2 stage was observed. In N-2, the cells grew from 4*10\hat{ }5 to almost 40*10\hat{ }5 cells/mL during a growth phase of three days. However, in the following N-1, the cells started at 7.8*10\hat{ }5 cells/mL and only reached 28.6*10\hat{ }5 cells/mL in the same time span.

The high growth rate of 0.79/d in N-2 was starting to decrease after the first day in N-1 and dropped drastically, ending up at only 0.47/d after three days. Simultaneously, the viability, which was sustained at a consistently high level in N-2, declined increasingly in N-1 from 97.5% to 92.4%. After the cell suspension was transferred to the main fermenter, the cells did not recover. Instead, the viability continued to decline from 94% to 86.9% before the planned transfection. Hence, no transfection could be performed. The viability continued to decline to 70% after four days, as did the growth rate, which dropped to 0.14/d by the end of the process.

There was no limitation at the end of the N-2 fermenter. Moreover, the addition of fresh medium with the transfer to the N-1 fermenter increased the substrate concentrations back to their initial value. However, glucose limitation occurred in N-1 at the end of the process with a concentration of 0.4 g/L. Without being bound by this theory, another reason for poor cell growth could be the high production and accumulation of waste products. During N-2, already high amounts of lactate and ammonium were accumulated. This led to the fact that the starting concentrations in N-1 were already higher than in N-2. Thus, almost 5 g/L lactate and 52 mg/L ammonium were reached at the end of the N-2 stage.

Due to the low VCD at the end of N-1, the entire fermentation volume had to be transferred to the main fermenter and the starting volume was reduced to 50 L instead of 60 L to reach the desired inoculation density of 20*10\hat{ }5 cells/mL.

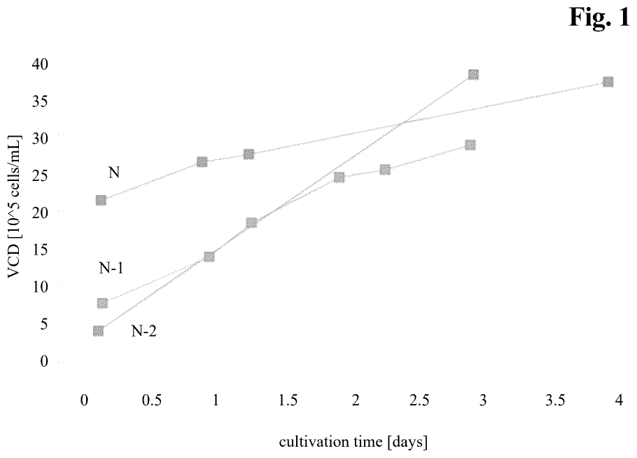

This can be seen in FIG. 1. Blue=N-2, fed-batch; green=N-1, fed-batch; red=production stage. The cells do not sufficiently grow in the cultivation, making a transfection for AAV production ineffective.

It has now been found that using perfusion in the propagation of the mammalian cells prior to AAV particle production according to the method according to the current invention resulted in an improvement, independently of whether the perfusion is a non-controlled or controlled perfusion.

In a first example according to the invention, using perfusion in the N-1 stage, fixed perfusion rates were set. The perfusion rates increased once per day from 0.21 vvd (day 1) over 0.42 vvd (day 2) to 0.63 vvd (day 3). Thus, a total of 29.1 L medium or 1.16 bioreactor volumes was fed to the cell culture during perfusion in this example.

However, the aeration control needed to be adjusted since the pO2 value fluctuated strongly and dropped to almost 0% directly after inoculation. In order to improve the oxygen input into the fermenter, the rocking rate was increased after 3.5 hours and further after 25.5 hours. The 02 controller used here was a proportional-integral-derivative (PID) controller. Its basic working principle is to read a sensor, calculate the difference between the desired set point (SP) and a measured process value (PV) and then apply a correction by setting the desired actuator output. This output is computed by calculating proportional, integral, and derivative responses and summing those three components. To reduce overshooting of the controller, the integral term was switched off after 6.5 hours. Only the proportional and derivative gains were used thereafter. The aeration rate was increased from 500 to 1000 mL/min after 26.5 hours.

The cells showed a higher cell growth up to a VCD of 92.3*10\hat{ }5 cells/mL from a starting VCD of 10.9*10\hat{ }5 cells/mL than in the comparative N-1 process without perfusion. Accordingly, compared to the comparative first run without perfusion a 3.2-fold higher cell density was reached with only 1.4-fold higher inoculation density. The viability remained at a high level throughout the whole run with a minimum of 96.9% one day after inoculation in contrast to the sharp decline in viability without perfusion. The growth rate was very high at the beginning with 1.05/d, but decreased during the process to 0.76/d after three days. Thus, the growth rate was between 1.3-fold higher at the beginning and 1.6-fold higher at the end than the growth rate in the comparative N-1 fermentation without perfusion. Thus, not only was the absolute growth rates increased but the decline in the growth rate in the process with perfusion was also reduced.

The cells resumed growth after a lag phase of 1.5 hours after inoculation in the main fermentation.

Cell growth in N-2 and N-1 was very good with final VCDs of 44*10\hat{ }5 cells/mL in N-2. In N-1, an even higher VCD of 92*10\hat{ }5 cells/mL was reached due to perfusion. Moreover, the viability constantly remained at high levels above 97% and final growth rates of 0.64/d in N-2 and 0.77/d in N-1 confirm the good cell proliferation in the inoculation train. This growth was continued at the beginning of the main fermenter with a growth rate of 0.80/d, which implies a successful transfer of the cell suspension from the N-1 into the N bioreactor.

As expected, the growth rate deteriorated after the transfection of the cell culture at day 1 to a final value of 0.12/d directly before harvesting on day 4. It should also be noted that the main fermentation was inoculated with a cell density of 26.77*10\hat{ }5 cells/mL instead of 20*10\hat{ }5 cells/mL. Thus, the cell density of 52*10\hat{ }5 cells/mL before medium addition was higher than the desired 36*10\hat{ }5 cells/mL.

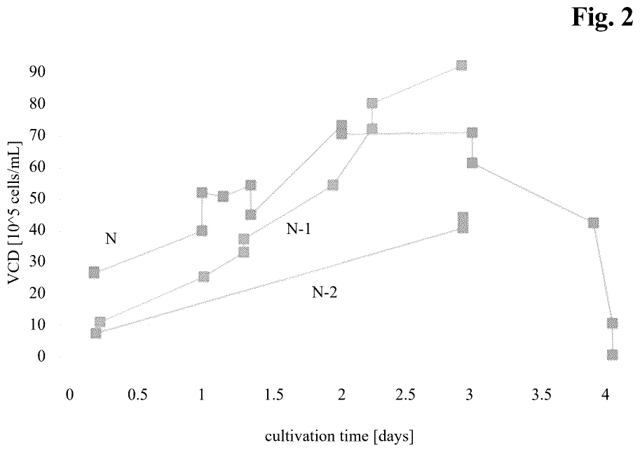

The results for the non-controlled perfusion in different cultivation volumes (scales) are shown in FIG. 2. Blue=N-2, fed-batch in 10 L scale; green=N-1, non-controlled perfusion in 25 L scale; red=production stage in 100 L scale. The perfusion rate was changed daily from 0.21 vvd to 0.42 vvd to 0.63 vvd. This resulted in consistent growth and high viabilities of the propagated cells. Production of an AAV particle of serotype 8 was successfully done.

However, despite the replenishment of nutrients by perfusion, glucose and glutamine concentrations declined like in the process without perfusion. Thus, substrate limitations occurred at the end of the fermentation. Without being bound by this theory, this could be explained by the fact that with perfusion significantly higher cell densities were achieved, with more cells also consuming higher absolute substrate amounts. This implies that the applied perfusion rates were not sufficient to supply enough nutrients to keep their concentration constant in a fast-growing cell culture. Therefore, the perfusion rates were adjusted to reduce or even eliminate the depletion of substrates. Therefore, a viable-cell-density-controlled (VCD-controlled) perfusion was employed.

Therefore, the perfusion rate was calculated as outlined in the examples section. The mean specific growth rate from the first example according to the invention with 0.764/d was used to plan the VCD. The goal for the N-1 fermentation was to reach a final VCD of approximately 60*10\hat{ }5 cells/mL in a 25 L working volume to provide enough cells for the inoculation of the main fermenter. Consequently, the working volume of the N-1 fermentation could be reduced while still achieving the required cell number with higher cell densities. If the working volume were reduced to 10 L, a final cell density of at least 120*10\hat{ }5 cells/mL would be required. According to the growth rate of 0.764 from the first perfusion run, an inoculation density of 15*10\hat{ }5 cells/mL would be needed to reach a final cell density of 150*10\hat{ }5 cells/mL. A scale-down model with a 1 L working volume was used.

The cell-specific substrate consumption was determined according to the predicted VCD. The cell-specific consumption rates for glucose (7.50*10\hat{ }−10 g/cell/d) and glutamine (1.20*10\hat{ }−10 g/cell/d) were calculated as an average of two previous processes with HEK293 cells in the 10 L rocker without perfusion. These were then used to determine the perfusion rates required to provide enough substrates for this cell-specific consumption. Since the perfusion rate to keep the glutamine concentration constant was lower than that for glucose, the perfusion rate for glucose was used. The perfusion rate was increased once a day. To avoid nutrient limitations through temporary medium supply interruption or process variability, each daily rate was calculated using the predicted VCD at the end of the day.

The planned VCD was only slightly higher than the actually measured VCD reached at the end of the process. A growth rate of 0.673/d was determined, which is 0.9-fold lower than the predicted one. This led to a final cell density of 128*10\hat{ }5 cells/mL after 2.9 days. The perfusion rate was manually readjusted during the process using the daily measured substrate concentrations and VCD. The subsequently resulting perfusion rate coincides very well with the perfusion rate based on the process conditions. Overall, 2.36 L medium were required for perfusion, corresponding to 2.36 bioreactor volumes.

In the last process section did the VCD increase more in the run with the controlled perfusion rates, reaching a final value of 128*10\hat{ }5 cells/mL instead of 92.3*10\hat{ }5 cells/mL as in the first perfusion run. This trend is also reflected in the growth rate. In the first perfusion process, the growth rate declined during the process, especially between 2.2 and 2.9 days from 0.90/d to 0.77/d. In the process with controlled perfusion rates, the growth rate was overall lower with an average of 0.67/d, but remained relatively constant and even increased towards the end.

The viability also shows a slight decrease at the end of the first perfusion process while it remained constant or increased slightly towards the end in the perfusion process with controlled perfusion rates.

The controlled perfusion process with adjusted perfusion rates prevented the limitation of glucose and glutamine at the end of the cultivation. However, the substrate concentrations could not be kept constant and the lactate and glutamate concentrations were similar or slightly higher than in the first perfusion process.

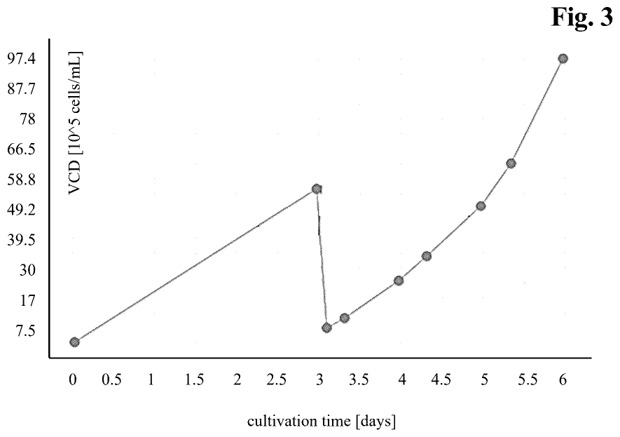

The results for viable-cell-density-controlled (VCD-controlled) perfusion in 11 scale are shown in FIG. 3 (controlled perfusion). The perfusion rate was calculated using cell specific consumption rates based on cell density measurements and calculation of cell growth. No substrate limitation was observed. To achieve the target cell density of 80-100*10\hat{ }5 cells/mL, a cell split was done after 3 days.

The prediction of the VCD using the growth rate from the previous perfusion process further improved the method according to the invention. The VCD prediction provided the necessary basis for the subsequent determination of the perfusion rate. With the controlled perfusion, it was not possible to keep the substrate concentration constant, but it prevented substrate limitation. This allowed consistently good cell growth and viability until the end of the process with constant growth rate. Compared to the first perfusion process, the controlled perfusion process used twice as much perfusion medium.

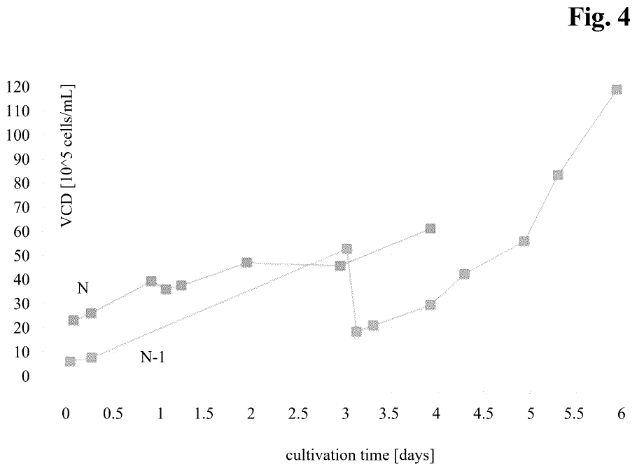

The results for VCD-controlled perfusion in 100 l scale are shown in FIG. 4. Green=N-1, controlled perfusion; red=production stage. Production of an AAV particle of subtype 2 variant 7m8 was successfully done.

The fermenter volume of the N-1 bioreactor was increased to 15 L to achieve a sufficiently high cell density for inoculation of the main fermenter. However, an inoculation VCD of approximately 13*10\hat{ }5 cells/mL was required to reach the target density of 100*10\hat{ }5 cells/mL after three days, resulting in a relatively high volume of pre-culture needed. Thus, the N-1 process was run for a longer period of time, in this example for six days. This would however require a very low inoculation VCD of 1.6*10\hat{ }5 cells/mL. Such low inoculation VCDs are difficult to set precisely since cell aggregates can lead to measurement inaccuracies. Even small deviations from this could lead to either a too low final VCD or an exorbitantly increased medium consumption since the medium consumption increases exponentially with the VCD. A solution to this problem is to inoculate with a higher, easily adjustable VCD and split the cell culture in the middle of the process, to reach the desired final VCD as exactly as possible. Consequently, the following perfusion process was inoculated with 5*10\hat{ }5 cells/mL and split to 13*10\hat{ }5 cells/mL on day 3, so that a final VCD of approximately 100*10\hat{ }5 cells/mL was reached after six days.

The cell culture was split after three days from 55.4*10\hat{ }5 to 10.5*10\hat{ }5 cells/mL resulting in a final VCD of 97.4*10\hat{ }5 cells/ml. A mean growth rate of 0.77/d in the first growth phase and 0.78/d in the second growth phase after the split was determined. This correlated well with the predicted average growth rate of 0.72/d from the previous two perfusion processes. Viability was 96% immediately after inoculation.

In total, 3.3 bioreactor volumes of perfusion medium were added throughout this process. No substrate limitation occurred during the first growth phase and the substrate concentrations remained even higher than in the previous perfusion processes. However, the inoculation VCD was lower with 5.8*10\hat{ }5 cells/mL compared to 17.4*10\hat{ }5 cells/mL in the first 1 L perfusion process.

While this new set-up of the N-1 fermenter did not shorten the overall duration of the inoculation train, it allowed the removal of the N-2 fermenter due to the lower inoculation density of N-1.

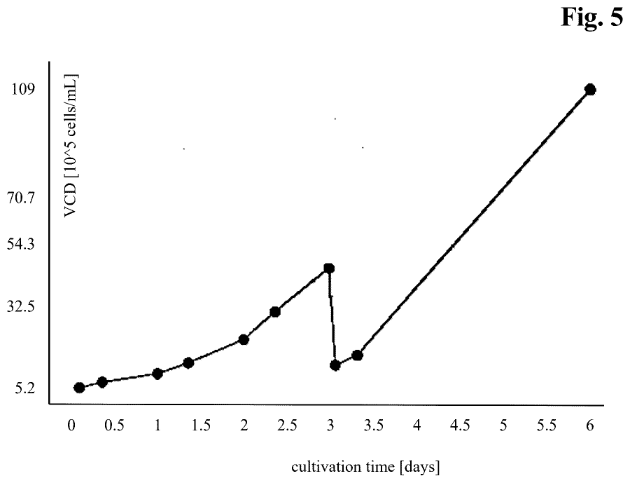

The process was repeated using a capacitance probe generated permittivity signal to control the perfusion rate including a split after propagation day 3. This resulted in a process according to the invention with automated perfusion control. A linear correlation between offline measured VCD and online measured permittivity was demonstrated. The found correlation of viable cell density and permittivity allowed a more economic control of the perfusion process resulting in a reduction of perfused cultivation medium by about 30% (3.3 reactor volumes to 2.2 reactor volumes). The cell density curve of the permittivity controlled perfusion process is shown in FIG. 5 (N-1, permittivity-controlled perfusion, 10 L scale). The applied cell specific perfusion rate was 107 pL/cell/day for this exemplary process. In other examples, a CSPR of 101 pL/cell/day has been applied.

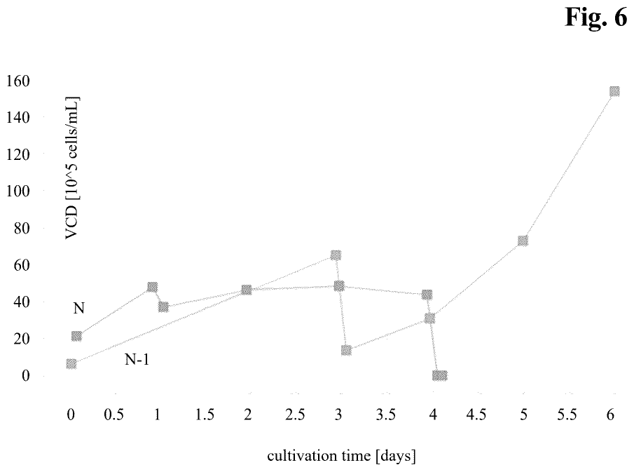

The process was repeated using an online-biomass sensor to control the perfusion rate including a split after propagation day 3. Production of an AAV particle of subtype 2 variant 7m8 comprising a transgene encoding a therapeutic Fab was successfully done. The cell density curve of the biomass-sensor-controlled perfusion process is shown in FIG. 6. Green=N-1, biomass-sensor-controlled perfusion; red=production stage.

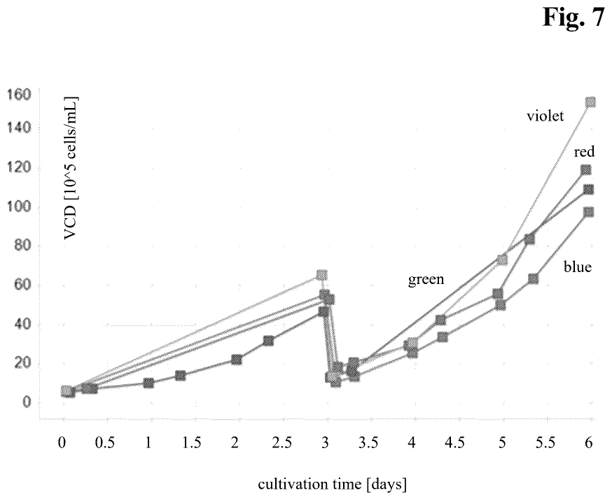

FIG. 7 shows two the viable cell densities of two pairs of VCD-controlled perfusion versus biosensor-controlled perfusion: red (VCD) versus violet (biosensor); blue (VCD) versus green (Biomass-sensor)).

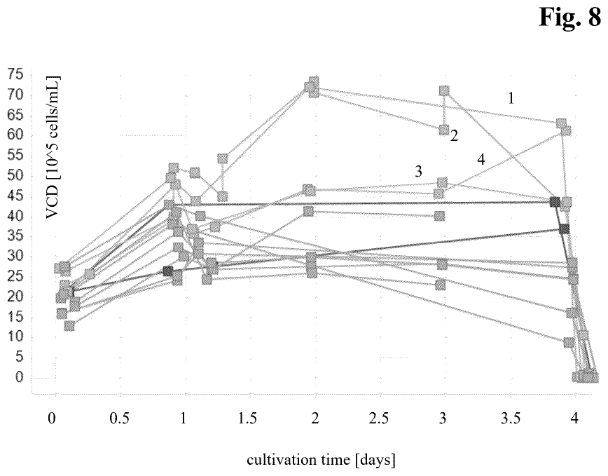

FIG. 8 shows the viable densities obtained in different production scale cultivations inoculated with cells obtained with perfusion in the N-1 stage, i.e. according to the invention, and without perfusion in the N-1 stage. The red curves correspond to production scale cultivation inoculated with cells from N-1 cultivations without perfusion and the green curves (marked as “1”, “2”, “3”, “4”) correspond to production scale cultivations inoculated with cells from N-1 cultivations with perfusion according to the invention. It can be seen that the viable cell densities obtained in the cultivation inoculated with cells propagated with perfusion in the N-1 stage is higher resulting in a higher AAV particle yield (transfection 24 hours post cultivation start).

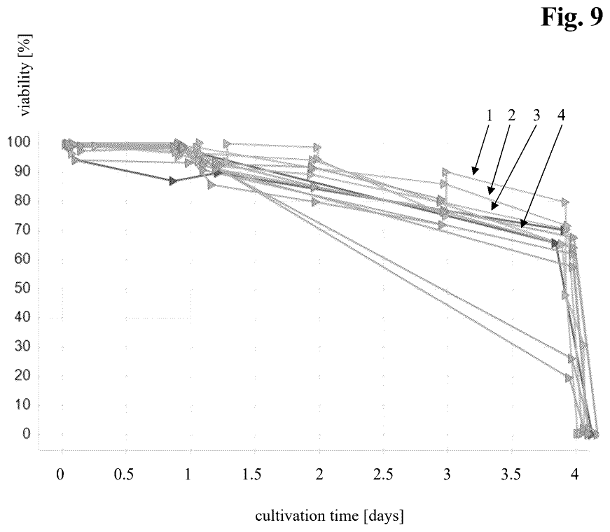

FIG. 9 shows the viability of the cells in the production scale cultivation N depending on the cultivation in the N-1 stage of the cells used for inoculation of the N stage. The red curves correspond to a production culture inoculated with cells obtained from a batch N-1 cultivation. The green curves (denoted as “1”, “2”, “3”, and “4”) correspond to production scale cultivations inoculated with cells obtained from N-1 cultivations with perfusion according to the current invention. It can be seen that that cells resulting from N-1 perfusion grow better and have a better response of the transfection after 24 hours post start, i.e. maintain higher viabilities. These cells still grow further and keep producing rAAV particles.

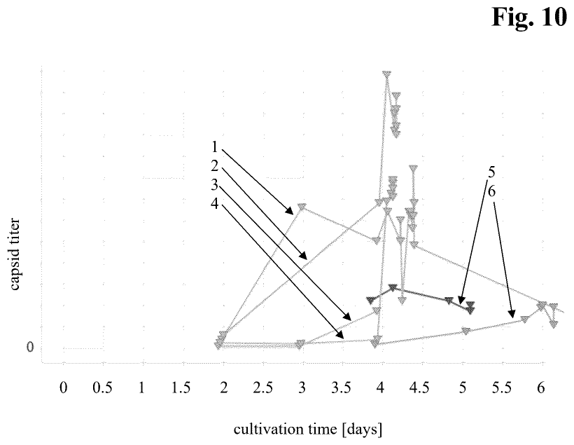

FIG. 10 shows the capsid titer in the production culture in bioreactor systems >10 L in N after N-1 with and without perfusion. The red curves (“5”, “6”) correspond to a production culture following a batch N-1. The green curves (“1”, “2”, “3”, and “4”) correspond to production scale cultures following N-1 with perfusion. For the red curves, batch culture N-1 was executed in the same bioreactor (therefore N scale cultivation day zero corresponds to day 4 in the figure). For comparable bioreactor systems, the effect of the increase of capsid titer using cells cultivated with perfusion in the N-1 stage for inoculation is significant.

Thus, the method according to the current invention achieves higher final cell density as well as capsid titer. The range of fluctuation is also reduced when the cells have been propagated using perfusion. Without being bound by this theory, it is assumed that cells propagated using perfusion have a higher tolerability with respect to the stress during transfection, recover faster, maintain higher viable cell densities and are still growing, although slowly.

All references mentioned herein are incorporated herewith by reference.

The following examples, sequences and figures are provided to aid the understanding of the present invention, the true scope of which is set forth in the appended claims. It is understood that modifications can be made in the procedures set forth without departing from the spirit of the invention.

### EXAMPLES

### General Techniques

### 1) Recombinant DNA Techniques

Standard methods are used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents are used according to the manufacturer's instructions.

### 2) DNA and Protein Sequence Analysis and Sequence Data Management

The EMBOSS (European Molecular Biology Open Software Suite) software package, Invitrogen's Vector NTI and Geneious Prime and are used for sequence creation, mapping, analysis, annotation and illustration.

### 3) Gene and Oligonucleotide Synthesis

Desired gene segments are prepared by chemical synthesis at Geneart GmbH (Regensburg, Germany). The synthesized gene fragments are cloned into an E. coli plasmid for propagation/amplification. The DNA sequences of subcloned gene fragments are verified by DNA sequencing. Alternatively, short synthetic DNA fragments are assembled by annealing chemically synthesized oligonucleotides or via PCR. The respective oligonucleotides are prepared by metabion GmbH (Planegg-Martinsried, Germany).

### 4) Reagents

All commercial chemicals, antibodies and kits are used as provided according to the manufacturer's protocol if not stated otherwise.

### 5) Cloning

### General

Cloning with R-sites depends on DNA sequences next to the gene of interest (GOI) that are equal to sequences lying in following fragments. Like that, assembly of fragments is possible by overlap of the equal sequences and subsequent sealing of nicks in the assembled DNA by a DNA ligase. Therefore, a cloning of the single genes in particular preliminary plasmids containing the right R-sites is necessary. After successful cloning of these preliminary plasmids the gene of interest flanked by the R-sites is cut out via restriction digest by enzymes cutting directly next to the R-sites. The last step is the assembly of all DNA fragments in one step. In more detail, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some plasmids, a cloning strategy via restriction enzymes was used. By selection of suitable restriction enzymes, the wanted gene of interest can be cut out and afterwards inserted into a different plasmid by ligation. Therefore, enzymes cutting in a multiple cloning site (MCS) are preferably used and chosen in a smart manner, so that a ligation of the fragments in the correct array can be conducted. If plasmid and fragment are previously cut with the same restriction enzyme, the sticky ends of fragment and plasmid fit perfectly together and can be ligated by a DNA ligase, subsequently. After ligation, competent E. coli cells are transformed with the newly generated plasmid.

### Cloning Via Restriction Digestion

For the digest of plasmids with restriction enzymes, the components shown in the following table are pipetted together on ice.

TABLE
Restriction Digestion Reaction Mix

| component | ng (set point) | μL |
|---|---|---|
| purified DNA | tbd | tbd |
| CutSmart Buffer (10x) | | 5 |
| Restriction Enzyme | | 1 |
| PCR-grade Water | | ad 50 |
| Total | | 50 |

If more enzymes are used in one digestion, 1 μL of each enzyme is used and the volume is adjusted by addition of more or less PCR-grade water. All enzymes are selected on the preconditions that they are qualified for the use with CutSmart buffer from new England Biolabs (100% activity) and have the same incubation temperature (all 37° C.).

Incubation is performed using thermomixers or thermal cyclers, allowing incubating the samples at a constant temperature (37° C.). During incubation the samples are not agitated. Incubation time is set at 60 min. Afterwards the samples are directly mixed with loading dye and loaded onto an agarose electrophoresis gel or stored at 4° C./on ice for further use.

A 1% agarose gel is prepared for gel electrophoresis. Therefor 1.5 g of multi-purpose agarose are weighed into a 125 Erlenmeyer shake flask and filled up with 150 mL TAE-buffer. The mixture is heated up in a microwave oven until the agarose is completely dissolved. 0.5 μg/mL ethidium bromide are added into the agarose solution. Thereafter the gel is cast in a mold. After the agarose is set, the mold is placed into the electrophoresis chamber and the chamber is filled with TAE-buffer. Afterwards the samples are loaded. In the first pocket (from the left), an appropriate DNA molecular weight marker is loaded, followed by the samples. The gel is run for around 60 minutes at <130 V. After electrophoresis, the gel is removed from the chamber and analyzed in an UV-Imager.

The target bands are cut and transferred to 1.5 mL Eppendorf tubes. For purification of the gel, the QIAquick Gel Extraction Kit from Qiagen is used according to the manufacturer's instructions. The DNA fragments are stored at −20° C. for further use.

The fragments for the ligation are pipetted together in a molar ratio of 1:2, 1:3 or 1:5 plasmid to insert, depending on the length of the inserts and the plasmid-fragments and their correlation to each other. If the fragment, that should be inserted into the plasmid is short, a 1:5-ratio is used. If the insert is longer, a smaller amount of it is used in correlation to the plasmid. An amount of 50 ng of plasmid is used in each ligation and the particular amount of insert calculated with NEBioCalculator. For ligation, the T4 DNA ligation kit from NEB is used. An example for the ligation mixture is depicted in the following Table.

TABLE
Ligation Reaction Mix

| component | ng (set point) | conc. [ng/μL] | μL |
|---|---|---|---|
| T4 DNA Ligase Buffer (10x) | | | 2 |
| Plasmid DNA (4000 bp) | 50 | 50 | 1 |
| Insert DNA (2000 bp) | 125 | 20 | 6.25 |
| Nuclease-free Water | | | 9.75 |
| T4 Ligase | | | 1 |
| Total | | | 20 |

All components are pipetted together on ice, starting with the mixing of DNA and water, addition of buffer and finally addition of the enzyme. The reaction is gently mixed by pipetting up and down, briefly microfuged and then incubated at room temperature for 10 minutes. After incubation, the T4 ligase is heat inactivated at 65° C. for 10 minutes. The sample is chilled on ice. In a final step, 10-beta competent E. coli cells are transformed with 2 μL of the ligated plasmid (see below).

### Transformation 10-Beta Competent E. Coli Cells

For transformation, the 10-beta competent E. coli cells are thawed on ice. After that, 2 μL of plasmid DNA is pipetted directly into the cell suspension. The tube is flicked and put on ice for 30 minutes. Thereafter, the cells are placed into a 42° C. thermal block and heat-shocked for exactly 30 seconds. Directly afterwards, the cells are chilled on ice for 2 minutes. 950 μL of NEB 10-beta outgrowth medium are added to the cell suspension. The cells are incubated under shaking at 37° C. for one hour. Then, 50-100 μL are pipetted onto a pre-warmed (37° C.) LB-Amp agar plate and spread with a disposable spatula. The plate is incubated overnight at 37° C. Only bacteria, which have successfully incorporated the plasmid, carrying the resistance gene against ampicillin, can grow on these plates. Single colonies are picked the next day and cultured in LB-Amp medium for subsequent plasmid preparation.

### Bacterial Culture

Cultivation of E. coli is done in LB-medium, short for Luria Bertani, which is spiked with 1 mL/L 100 mg/mL ampicillin resulting in an ampicillin concentration of 0.1 mg/mL. For the different plasmid preparation quantities, the following amounts are inoculated with a single bacterial colony.

TABLE
E. coli cultivation volumes

| | Volume LB-Amp | |
|---|---|---|
| Quantity plasmid preparation | medium [mL] | Incubation time [h] |
| Mini-Prep 96-well (EpMotion) | 1.5 | 23 |
| Mini-Prep 15 mL-tube | 3.6 | 23 |
| Maxi-Prep | 200 | 16 |

For Mini-Prep, a 96-well 2 mL deep-well plate is filled with 1.5 mL LB-Amp medium per well. The colonies are picked and the toothpick is tuck in the medium. When all colonies are picked, the plate is closed with a sticky air porous membrane. The plate is incubated in a 37° C. incubator at a shaking rate of 200 rpm for 23 hours.

For Mini-Preps a 15 mL-tube (with a ventilated lid) is filled with 3.6 mL LB-Amp medium and equally inoculated with a bacterial colony. The toothpick is not removed but left in the tube during incubation. Like the 96-well plate, the tubes are incubated at 37° C., 200 rpm for 23 hours.

For Maxi-Prep 200 mL of LB-Amp medium are filled into an autoclaved glass 1 L Erlenmeyer flask and are inoculated with 1 mL of bacterial day-culture, that is roundabout 5 hours old. The Erlenmeyer flask is closed with a paper plug and incubated at 37° C., 200 rpm for 16 hours.

### Plasmid Preparation

For Mini-Prep, 50 μL of bacterial suspension are transferred into a 1 mL deep-well plate. After that, the bacterial cells are centrifuged down in the plate at 3000 rpm, 4° C. for 5 min. The supernatant is removed and the plate with the bacteria pellets is placed into an EpMotion. After approx. 90 minutes, the run is done and the eluted plasmid-DNA can be removed from the EpMotion for further use.

For Mini-Prep, the 15 mL tubes are taken out of the incubator and the 3.6 mL bacterial culture is splitted into two 2 mL Eppendorf tubes. The tubes are centrifuged at 6,800×g in a tabletop microcentrifuge for 3 minutes at room temperature. After that, Mini-Prep is performed with the Qiagen QIAprep Spin Miniprep Kit according to the manufacturer's instructions. The plasmid DNA concentration is measured with Nanodrop.

Maxi-Prep is performed using the Macherey-Nagel NucleoBond® Xtra Maxi EF Kit according to the manufacturer's instructions. The DNA concentration is measured with Nanodrop.

### Ethanol Precipitation

The volume of the DNA solution is mixed with the 2.5-fold volume ethanol 100%. The mixture is incubated at −20° C. for 10 min. Then the DNA is centrifuged for 30 min. at 14,000 rpm, 4° C. The supernatant is carefully removed and the pellet is washed with 70% ethanol. Again, the tube is centrifuged for 5 min. at 14,000 rpm, 4° C. The supernatant is carefully removed by pipetting and the pellet is dried. When the ethanol is evaporated, an appropriate amount of endotoxin-free water is added. The DNA is given time to re-dissolve in the water overnight at 4° C. A small aliquot is taken and the DNA concentration is measured with a Nanodrop device.

### Expression Cassette Composition

For the expression of an open reading frame, a transcription unit comprising at least the following functional elements is used:
a promoter,
a nucleic acid comprising the respective open reading frame including signal sequences, if required,
a polyadenylation signal sequence.

Beside the expression unit/cassette including the desired gene to be expressed, the basic/standard mammalian expression plasmid contains
an origin of replication from the plasmid pUC18 which allows replication of this plasmid in E. coli, and
a beta-lactamase gene which confers ampicillin resistance in E. coli.

### 6) Cell Culture Techniques

Standard cell culture techniques are used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Transient Transfections in HEK293 System

Cells producing a recombinant AAV particle have been generated by transient transfection with the respective plasmids using the HEK293 system (Invitrogen) according to the manufacturer's instruction. Briefly, HEK293 cells (Invitrogen) growing in suspension either in a shake flask or in a stirred fermenter in serum-free FreeStyle™ 293 expression medium (Invitrogen) are transfected with a mix of the respective plasmids and 293fectin™ or fectin (Invitrogen). For 2 L shake flask (Corning) HEK293 cells are seeded at a density of 1*10⁶ cells/mL in 600 mL and are incubated at 120 rpm, 8% CO₂. The day after the cells are transfected at a cell density of ca. 1.5*10⁶ cells/mL with ca. 42 mL mix of A) 20 mL Opti-MEM (Invitrogen) with 600 μg total plasmid DNA (1 μg/mL) and B) 20 mL Opti-MEM+1.2 mL 293 fectin or fectin (2 μL/mL). According to the glucose consumption, glucose solution is added during the course of the fermentation.

### 7) Monitoring and Analytical Methods

### Dielectric Spectroscopy

Dielectric Spectroscopy in the BioStat® RM 20/50 Rocker was performed using the pre-installed single-use viable biomass sensor BioPAT® ViaMass. Before starting the fermentation, a signal test with a low (0 pF/cm, 0 mS/cm) and a high (100 pF/cm, 40 mS/cm) signal simulator was conducted prior to connecting the probe with the associated port on the bag.

For the HyClone® bioreactor, the Incyte system was used. The IncyteArc 220 probe was inserted into the probe assembly sheath, autoclaved, and installed into the bioreactor.

Before inoculation, the permittivity probes were zero adjusted to the medium following the manufacturer's instruction. For the IncyteArc 220, the ArcAir application was used for zero adjustment. Both systems measure capacitance, which is computed as permittivity ε in pF/cm. The permittivity correlates with the VCV or VCD linearly and therefore enables the estimation of the viable cell density in real-time.

### Cell Culture Analysis

The VCD, viability, average cell diameter, as well as the aggregation rate, were measured using the Cedex® HiRes analyzer. It is a fully automated, image-based cell analyzer using the trypan blue exclusion method in combination with a high-resolution image scanner.

For the determination of the above-mentioned cell parameters, a predefined sample volume of cell suspension was aspirated via a syringe module and manifold valve and mixed with the required amount of 0.2% trypan blue solution. Cells with non-intact cell membranes were stained blue by the diffusion of the dye into the cell interior. In intact cells, the dye could not enter. The dyed cell suspension was transferred to a flow chamber via capillary tubing and the manifold valve. After a fixed sedimentation time, a digital image using a scanner was taken. The scanned image of the flow chamber was divided into smaller images for evaluation and analyzed using analysis software on the associated computer.

Depending on the estimated total cell density (TCD), dilution of the process sample with PBS according to the following table was necessary prior to an analysis to be in the working concentration range of the system (0.1-50 10\hat{ }5 cells/mL).

TABLE
Required dilutions for the measurement with the Cedex ® HiRes
analyzer according to the estimated total cell density.

| Total cell density [10\hat{ }5 cells/mL] | dilution |
|---|---|
| 0-50 | none (1:1) |
| 51-100 | 1:2 |
| 101-200 | 1:5 |
| 201-500 | 1:10 |
| >500 | 1:20 |

Besides typical cell parameters, the Cedex® HiRes analyzer provides information about aggregates and morphological parameters. Information about the aggregation of the cells was relevant for this work since the cell line used is prone to aggregation, which may reduce the transfection success (Girard et al., P., Cytotechnol. 38 (2002) 15-21). The Cedex® HiRes software determines the cells that are in aggregates with at least one other cell and calculates the aggregation rate as the percentage of cells found in aggregates relative to the total cell count.

The relative standard deviation for multiple measurements with the Cedex® HiRes analyzer is given as 5%. However, due to additional dilution errors when exceeding the measuring range, a relative standard deviation of 6% can be assumed according to internal specifications.

Apart from the directly measured cell parameters, the viable cell volume (VCV) was calculated since it was needed for a correlation with the permittivity signal from the capacitance probe. For the calculation of the viable cell volume, a spherical cell shape was assumed. With the VCD in cells/mL and the average diameter of the cells (d) in m measured with the Cedex® HiRes analyzer, the VCV in mm3/mL was determined according to equation (14):

$$VCV = VCD * \frac{4}{3} * π * {( \frac{d}{2} )}^{2} \qquad ( 14 )$$

### EXAMPLE 1—REFERENCE EXAMPLE

Basic Process for rAAV Particle Production without Perfusion

The production of rAAV particles was started with the inoculation of a 125 mL shake flask with a frozen cell aliquot after thawing. Every three to four days, the cell culture was passaged and the volume was increased. After a sufficient volume, i.e. cell density, for the inoculation of the N-2 bioreactor was reached, the pre-culture was transferred to a 10 L wave-mixed bioreactor with an inoculation density of about 5*10\hat{ }5 cells/mL and cultivated for three days. Subsequently, after reaching a cell density of approximately 40*10\hat{ }5 cells/mL, the cell culture was further transferred to a 25 L wave-mixed bioreactor, with an inoculation density of about 8*10\hat{ }5 cells/mL. In both wave-mixed bioreactors, only base was added. Afterward, the cell suspension was used to inoculate the main fermenter with a starting volume of 60 L and a target inoculation density of 20*10\hat{ }5 cells/mL. After 24 hours an additional volume of 20% fresh cultivation medium based on the actual cultivation volume was added. Thereafter the cells were transfected with the respective plasmids. After a total of four days, the production process was terminated and lysis started by adding lysis buffer, nuclease, and alkaline solution. Subsequently, the lysed cell solution was transferred to a harvest tank and filtered to harvest rAAV particles.

### EXAMPLE 2

### Biostat® RM Rocker Operation

Small-scale experiments, as well as N-2 and N-1 seed train fermentations, were performed in Biostat® RM Rockers comprising a wave-mixed benchtop bioreactor and a Biostat® B control tower. These bioreactors utilize rocking motion mixing technology and are used with single-use Flexsafe® RM bags. For N-2 fermentations in 10 L scale, the Biostat® CultiBag RM 50 was used. N-1 fermentations in 15 L or 25 L scale and 1 L small-scale perfusion experiments were performed with the Biostat® RM 20/50 basic. Both have an exchangeable bag holder to fit bags with a total volume of 1-50 L. The rocking platform comprises an integrated local controller, air- and CO2-mixing module, and load cells.

The wave generated in the RM bags ensures effective gas exchange through the gas-liquid interface via two mechanisms, namely surface aeration and air entrainment by the breaking wave. For sufficient aeration, four different gas lines for compressed air, O2, N2, and CO2 are equipped with flow meters and four mass flow controllers were used. An integrated pressure sensor continuously measures the pressure inside the bag and sets the aeration to achieve a slight overpressure. The Flexsafe® RM bags are equipped with inlet and exhaust air filters.

Two heating elements using electric resistance heating are integrated directly into the bag holder and can be controlled separately. For small-scale experiments, only the left heating circuit was used and the 1 L bags were placed on the left side of the bag holder. A Pt100 resistance thermometer in the bag holder measures the bag's temperature. As the heating of the Biostat® RM produces slight condensation on the exhaust filter, a filter heater keeps the filter dry and prevents it from being blocked. A hood serves as a safety cover with an opening on the front for bag handling. It protects the attached bag from mechanical influences during operation and reduces heat loss.

The Flexsafe® RM bags are equipped with single-use opto-chemical pH and pO2 probes, and, where required, viable biomass probes (BioPAT® ViaMass). The pH controller regulated the addition of sodium carbonate and CO2 gas flow. Furthermore, the bags have free ports with C-Flex tubing and Luer or MPC connectors for the addition of media, inoculum, and base. A dip tube with a Luer septum is used for sampling.

For perfusion experiments, special bags with an integrated 1.2 m perfusion membrane were used. The membrane was fixed at the bottom of the bag, forming a compartment for the removal of cell-free media. The surface of the membrane is flushed with every rocking motion by the thereby created wave, which ensures low fouling and long cultivation times. Medium addition and permeate removal are performed using the peristaltic pumps on the Biostat® B control tower. The medium bag was placed on a scale to enable gravimetric control.

### EXAMPLE 3

### 100 L Single-Use Bioreactor HyClone® Operation

The main fermentation with the purpose to produce rAAV particles was performed in the stirred tank Single-Use Bioreactor HyClone® with a total working volume of 100 L. It included a stainless steel fermenter vessel, a Single-Use BioProcess® Container (BPC), an operating station with a DCU, a temperature control unit, as well as a medium station with six scales and peristaltic pumps. The reactor was equipped with a double jacket for temperature control and a lengthwise view glass. Temperature control was conducted by the temperature control unit. A pitched 3-blade impeller was used for stirring.

The aeration was performed via a submerged aeration tube with a ring-sparger and an open pipe. The BPC was equipped with two inlet air filters at the bottom, two exhaust air filters, and vent filter heaters at the top. Besides the exhaust air port at the top of the BPC, there was an inlet for headspace aeration, a pressure sensor as well as several free ports for the addition of antifoam, base, and feeds. The BPC enclosed two lateral port levels in the lower third of the bag. The lower port level comprised one port for the temperature probe and four AseptiQuik® connector probe ports, which were used to install the pH-, pO2- and capacitance probe. A built-in dip tube was used for sampling. Above these connectors, the BPC featured a free port with C-Flex tubing which was used for fresh medium addition and transfection. For harvesting, the BPC had a drain line at the bottom.

Bags with base as well as different feeds were placed on hanging scales at the medium station to enable gravimetric control. The pH controller regulated the base addition. Antifoam was added as required.

### EXAMPLE 4

### Pre-Culture and Inoculation

The pre-culture was performed in shake flasks with the fermentation medium. Cells were split every three to four days to 5*10\hat{ }5 cells/mL until they reached a cell density of approximately 40 to 60*10\hat{ }5 cells/mL while increasing the culture volume to the required amount for inoculation. During this time, the shake flasks were incubated at 37° C., 85% humidity, 5% CO2, with shaking.

For the 100 L fermentations in a HyClone® bioreactor, cells were further cultivated in N-2 and N-1 Biostat® RM Rockers before they were used to inoculate the main fermenter.

The inoculation procedure for fermentations in the Biostat® RM Rocker as well as in the HyClone® bioreactor was comparable. At first, the inoculum volume was determined dependent on the current and the target initial VCD. The pre-culture was pumped into the bioreactor using a low pump speed preventing excessive shear stress.

For large pre-culture volumes needed to inoculate the 100 L HyClone® bioreactor, the cells were transferred gently and quickly into the fermenter using hydrostatic pressure difference.

### EXAMPLE 5

### Transfection Procedure

Triple transfection was performed 24 hours after inoculation at a target VCD of 36*10\hat{ }5 cells/mL.

Directly before transfection, 20% (v/v) of the initial culture volume was added as fresh medium to the bioreactor. The transfection reagents (PEI MAX®) were prepared, mixed with the plasmids and filled up with fermentation medium. After mixing, the solution was transferred to a 10 L FLEXBOY® bag for incubation. At the end of the incubation period, the content of the bag with the transfection complexes was transferred into the bioreactor. Subsequently, the free PEI solution and then a VPA solution were pumped into the bioreactor to reach a final concentration of 5 mM VPA.

### EXAMPLE 6

### Perfusion Bioreactor Operation

The pH set point was defined and the dead band was set to 0.02 pH units. For pH control, a two-sided control loop was used in which CO2 sparging or 1 M sodium carbonate solution addition (in the following referred to as base) was applied to decrease or increase pH, respectively.

### 1) Manual Operation of the Perfusion Process—Daily Change

Fixed perfusion rates based on the amount of medium remaining from the previous process were used. The perfusion rate was increased gradually once a day.

### 2) Manual Operation of the Perfusion Process—Based on Cell-Specific Rates

Fixed perfusion rates were used, which were adapted to the cell-specific substrate consumption.

Cell-specific substrate consumption rate were determined based on previous cultivations and multiplied with the predicted VCD to calculate the amount of substrate required by the cells.

Prediction of VCD with Specific Growth Rates

The VCD was predicted with the mean specific growth rate over the entire process time calculated in the first perfusion cultivation above. The mean specific growth rate was determined by plotting the natural logarithm of the VCD against the process time t and inserting a linear fit with the absolute value of the slope being the specific growth rate y according to the following equation (Zhang, X., et al., “1.21—Modes of Culture/Animal Cells”, in Comprehensive Biotechnology (Second Edition), M. Moo-Young, (Ed.) Burlington: Academic Press, 2011, pp. 285-302):

$$ln ( VCD ( t ) ) = μ * t + b \qquad ( 2 )$$

This leads to a simplification, because the specific growth rate can fluctuate throughout the process. However, since a perfusion process typically allows for very stable cell growth, the mean growth rate can be used to predict the VCD. Thus, the course of VCD for the next perfusion experiment could be predicted by using equation (3):

$$VCD ( t ) = V C {D}_{initial} * {e}^{μ * t} \qquad ( 3 )$$

### Calculation of Cell-Specific Consumption Rates

Simultaneously, the cell-specific consumption rates were calculated from previous N-2 fermentations since no substrate was added or removed except by the cellular metabolism, which simplified the calculation. Moreover, the substrate concentrations instead of their absolute weight could be used for calculating the consumption rates, since the fermenter weight remained constant. The cell-specific consumption rate is defined as the amount of consumed substrate by a single cell in unit time, usually measured in pg/cell/d. The calculation involves the integral of viable cell density (IVCD), which is understood as the production capacity of the cell culture in cell*d/mL (see Zhang, supra). Thus, the cell-specific consumption rate q_{cons} was determined by taking the difference in substrate concentrations at the end cₜ and beginning of the fermentation cₜ₋₁ and dividing it by the difference in the associated IVCD according to equation (4):

$${q}_{cons} = \frac{{c}_{t} - {c}_{t - 1}}{{IVCD}_{t} - {IVCD}_{t - 1}} . \qquad ( 4 )$$

With IVCD defined as

$$IVCD = {{\int }^{t} VCD dt \approx {IVCD}_{t - 1} + \frac{{VCD}_{t - 1} + {VCD}_{t}}{2}}_{t - 1} * Δ t \qquad ( 5 )$$

Next, the absolute consumption rate q_{cons,abs} in g/L/d was predicted by multiplying the predicted VCD with the determined consumption rates.

$${q}_{cons , abs} = VCD * {q}_{cons}$$

### Determination of Perfusion Rates

From this, the perfusion rate pvvd in vvd could be obtained by dividing the absolute consumption rate q_{cons,abs} in g/L/d by the concentration of substrate in the medium c_{medium} in g/L with equation (7).

$${p}_{vvd} = \frac{{q}_{cons , abs}}{{c}_{medium}} \qquad ( 7 )$$

Moreover, the maximum perfusion rate was used by calculating each daily rate using the maximum predicted VCD to allow for fluctuations in substrate consumption. To enable a comparison between different perfusion processes, the cell-specific perfusion rate CSPR in pL/cell/d was also calculated according to Bausch et al. (Biotechnol. J. 14 (2019) 1700721). Therefore, the perfusion rate pvvd in vvd was divided by the VCD:

$$CSPR = \frac{{p}_{vνd}}{VCD} \qquad ( 8 )$$

A scale-down model was established in Flexsafe® RM bags with 1 L working volume. This was a physically much smaller version, albeit under the same conditions. The perfusion rates were further adjusted by manual daily sampling based on trypan blue exclusion cell counting and the determination of the substrate and metabolite concentrations as described below. The perfusion rates were adjusted if the VCD was higher than expected or the substrate concentrations dropped too much. In addition, the calculation of the predicted VCD and substrate consumption was refined with each experiment by using mean values of the specific growth rate and the cell-specific substrate consumption rate instead of individual values for further calculations.

Perfusion Automation with a Capacitance Probe

For automation of the perfusion rate, a robust automatic perfusion rate control system based on the live cell concentration from an online capacitance probe was established. The system operated in a completely closed loop so that no samples needed to be taken to obtain process information. In the control algorithm, a CSPR is specified and the permittivity signal of the capacitance probe is converted into a perfusion rate through calculation and implementation with a variable speed controlled pump.

To achieve this, the permittivity output signal of the probe was converted to the online VCD. Therefore, a correlation between the offline measured VCD and the permittivity signal was determined. To determine the correlation factor c_{VCD,Perm}, the VCD was plotted against permittivity ε and a linear trend line was inserted according to equation (9):

$$VCV = {c}_{VCD , Perm} * ε \qquad ( 9 )$$

To determine the pump output necessary to replenish the required volume of feed medium based on the current permittivity signal and the previously determined CSPR, the VCD was predicted and the required perfusion rate pvvd was calculated with the above-described equations. The correlation between the perfusion rate pvvd and the required pump output pumpout in % was established with the data of previous perfusion experiments. The associated correlation factor c_{pump,pvvd} was determined by inserting a linear fit using the following equation:

$${pump}_{out} = {c}_{pump , {p}_{vvd} * {p}_{vvd}} \qquad ( 10 )$$

With this correlation factor c_{pump,pvvd}, the pump output was determined from the required perfusion rate pvvd. To enable the automatic control of the pump via the permittivity signal E, the automation factor ƒₐᵤₜₒ was calculated with equation (11) and entered into the control software of the bioreactor:

$${f}_{auto} = \frac{{pump}_{out}}{ε} \qquad ( 11 )$$

This allowed the pump output to be set by multiplying the automation factor with the permittivity signal. In addition to the control loop for the perfusion feed, another control loop for the removal of permeate based on the bioreactor weight was used. This is a control loop, triggering the cell-free permeate outflow to harvest. Since the bioreactor was placed on a scale, the permeate pump was configured to start removing medium as soon as the bioreactor weight was greater than the initial weight directly after inoculation, until the weight was again at the starting point.

To assess the accuracy of the VCD prediction, the mean absolute percentage error (MAPE) was determined. This is a measure to assess the overall performance of a prediction model. As described in equation (12), the MAPE in % was determined by the sum of deviations of predicted values xpred from actual values xi normalized to the actual value, which was divided by the number of data points n.

$$MAPE = \frac{1}{n} * {\sum }_{i = 1}^{n} \vert \frac{{x}_{l} - {x}_{pred}}{{x}_{i}} \vert * 100 \% \qquad ( 12 )$$

### EXAMPLE 7

### Evaluation of Cell Growth and Metabolism

The specific growth rate μ represents the dynamic behavior of the cells and provides information about their growth and thus, indirectly, the health of the cell culture. It is a good basis for comparison for different processes (A. K. Srivastava and S. Gupta, “2.38—Fed-Batch Fermentation—Design Strategies”, in Comprehensive Biotechnology (Second Edition), M. Moo-Young, (Ed.) Burlington: Academic Press, 2011, pp. 515-526). The course of the growth rate over the entire fermentation was determined by dividing the difference between the logarithmized VCD and the inoculation VCD (VCD₀) by the corresponding time difference (Zhang, X., et al., “1.21—Modes of Culture/Animal Cells”, in Comprehensive Biotechnology (Second Edition), M. Moo-Young, (Ed.) Burlington: Academic Press, 2011, pp. 285-302).

$$μ = \frac{ln ( {VCD}_{n} ) - ln ( {VCD}_{0} )}{( {t}_{n} - {t}_{0} )} \qquad ( 15 )$$

Thus, the last growth rate in the process also corresponds to the mean growth rate over the entire process time. For fermentations with a split of the cell culture, VCD₀ was set to the VCD directly after the split.

The cell-specific formation and consumption rates can be determined according to equation (4) in the absence of feeding/perfusion and with the following equation (16) in perfusion processes:

$${q}_{form / cons} = \frac{{m}_{form / cons}}{( {IVCD}_{n} - {IVCD}_{n - 1} ) * {V}_{f 1}} = \frac{{m}_{out} - {m}_{in} + {m}_{n} - {m}_{n - 1}}{( {IVCD}_{n} - {IVCD}_{n - 1} ) * {V}_{f 1}} \qquad ( 16 )$$

$$with$$

$${m}_{out} = \frac{{c}_{n - 1} + {c}_{n} + \frac{{m}_{bolus}}{{V}_{f 1}}}{2} * p * ( {t}_{n} - {t}_{n - 1} ) \qquad ( 17 )$$

$$and$$

$${m}_{in} = {c}_{med + feed} * p * ( {t}_{n} - {t}_{n - 1} ) + {m}_{bolus} \qquad ( 18 )$$

wherein the abbreviations are as follows:

| | | | |
|---|---|---|---|
| c_{med+feed} | Concentration in | mₙ₋₁ | Mass in fermenter at |
| | medium + feed | | tₙ₋₁ |
| cₙ₋₁ | Concentration in | mₙ | Mass in fermenter at |
| | fermenter at tₙ₋₁ | | tₙ |
| cₙ | Concentration in | mₒᵤₜ | Discharged mass |
| | fermenter at tₙ | | between tₙ₋₁ and tₙ |
| IVCDₙ₋₁ | Integral of viable cell | m_{form/cons} | Formed/consumed |
| | density at tₙ₋₁ | | mass |
| IVCDₙ | Integral of viable cell | p | Perfusion rate in l/d |
| | density at tₙ | | |
| m_{bolus} | Mass from bolus | q_{form/cons} | Cell-specific formation/ |
| | added between tₙ₋₁ | | consumption rate |
| | and tₙ | | |
| mᵢₙ | Added mass between | V_{f1} | Fermenter volume |
| | tₙ₋₁ and tₙ | | |

While the metabolite production rate is positive, the substrate consumption rate is negative since the substrate is removed from the culture medium and thus the amount in the medium is decreasing.

1. A method for producing a recombinant AAV particle comprising the following steps:
a) propagating a mammalian cell using perfusion until at least a first pre-determined cell density is achieved;
b) diluting a fraction of the cells obtained in step a) by adding fresh cultivation medium to obtain a production cell solution that has a second pre-determined cell density;
c) cultivating the production cell solution for 1 to 36 hours;
d) transfecting the cells directly in the cultivated production cell solution obtained in step c) with one or more nucleic acids encoding for the recombinant AAV particle;
e) cultivating the transfected production cell solution obtained in step d) for 24 to 144 hours;
thereby producing a recombinant AAV particle.

2. The method of claim 1, wherein the first pre-determined cell density is at least 80*10\hat{ }5 cells/mL.

3. The method of claim 1, wherein the mammalian cell is a CHO-K1 or a HEK293 cell.

4. The method of claim 1, wherein the one or more nucleic acids comprise
i) a transgene comprising in 5′- to 3′-direction
alpha) a first ITR sequence;
beta) a promoter;
gamma) a nucleic acid sequence encoding a therapeutic molecule;
delta) a polyadenylation signal sequence;
epsilon) a second ITR sequence;
ii) a rep open reading frame;
iii) a cap open reading frame; and
iv) adenoviral E1A, E1B, E2A, E4orf6, and VA RNA open reading frames.

5. The method of claim 1, wherein in step b) 4-times to 6-times the volume of the aliquot of fresh cultivation medium is added.

6. The method of claim 1, wherein the second pre-determined cell density is about 20*10\hat{ }5 cells/mL.

7. The method of claim 1, wherein the cultivating in step c) is for about 24 hours.

8. The method of claim 1, wherein the cultivating in step e) is for about 72 hours.

9. The method of claim 1, wherein step e) is without feeding.

10. The method of claim 1, wherein the method further comprises the steps of
f) harvesting the cells from the cultivated solution of step e);
g) lysing the cells obtained in step f);
h) separating the AAV particles from the lysed cells obtained in step g);
i) optionally purifying the AAV particles.

11. The method according to claim 1, wherein the cell density after step c) is 35-40*10\hat{ }5 cells/ml.

12. The method according to claim 1, wherein after step c) and prior to step d) the following step cd) is performed:
cd) adding an additional about 20% of the cultivation volume fresh medium.

13. The method according to claim 1, wherein the transfecting is by adding the one or more nucleic acids as a mixture of PEI-complexed nucleic acid and free PEI.

14. The method according to claim 1, wherein the transfecting is in the presence of a final concentration of 5 mM valproic acid.
